(19) United States
(12) Patent Application Publication
(10) Pub. No.: US 2008/0237544 A1
(43) Pub. Date: Oct. 2, 2008

(54) Thermally Modulated Antioxidants

(76) Inventor: Juan C. Scaiano, Ottawa (CA)

Correspondence Address: KIRBY EADES GALE BAKER, BOX 3432, STATION D, OTTAWA, ON, K1P 6N9, CA

(21) Appl. No.: 10/586,734

(22) PCT Filed: Jan. 21, 2005
(86) PCT No.: PCT/CA2005/000068

§ 371 Date: Mar. 26, 2007

Related U.S. Application Data
(60) Provisional application No. 60/537,882, filed on Jan. 22, 2004.

(51) Int. Cl. C07D 307/83; C07C 13/547; C07C 69/76; C07C 255/32; C09K 15/06

(52) U.S. Cl. 252/407; 549/307; 585/16; 560/80; 558/426

(57) ABSTRACT

Most carbon-centered free radical antioxidants are generated through hydrogen abstraction. Disclosed herein a new class of antioxidant precursor compounds of the formula A-B, wherein upon exposure of the compounds to an increase in temperature, the compounds undergo at least partial dissociation into free radicals Ao and Bo at least one of which may be functional as an antioxidant. Preferably, each of Ao and Bo is a carbon centered free radical.

### FIELD OF THE INVENTION

The present invention relates generally to compounds that function as antioxidants. In particular, the present invention pertains to activatable antioxidant precursor compounds, methods for generating antioxidants and methods for preventing or reducing oxidation.

### BACKGROUND TO THE INVENTION

Antioxidants, or more specifically, chain breaking antioxidants are able to stop, delay or slow down the oxidative degradation of materials. In general, the structure of antioxidants is such that it confers on these molecules two key properties:
(a) they are generally good hydrogen donors, and (b) the radical produced following hydrogen transfer is unreactive toward oxygen, thus being able to stop the chain reaction responsible for autooxidations. In the case of some antioxidants the radical
(b) is capable of scavenging a second chain carrier, thus leading to a 2-1 stoichiometry; such is the case for phenolic antioxidants.

Requirement (b), i.e. lack of reactivity toward oxygen has resulted in many antioxidants containing O—H bonds, since the resulting oxygen centered radicals are frequently unreactive towards molecular oxygen.

In the last few years a few antioxidants have been proposed that do not contain labile O—H bonds, such as Irganox HP-136™ commercialized by Ciba Speciality Chemicals. Related antioxidants can be reviewed, for example, in U.S. Pat. No. 5,367,008 issued Nov. 22, 1994, and U.S. Pat. No. 5,428,177, issued Jun. 27, 1995, which are incorporated herein by reference.

Peroxyl free radical antioxidants and carbon centered free radical antioxidants are also discussed and disclosed in Scaiano, J. C. et al. “A Carbon-Centered Radical Unreactive Toward Oxygen: Unusual Radical Stabilization by a Lactone Ring”, (1999), Organic Letters, 2(7), 899-901; Bejan, E. V. et al “Lactone-Derived Carbon-Centered Radicals: Formation and Reactivity with Oxygen”, (2001), Organic Letters, 3(25), 4059-4062; Font-Sanchis, E. et al. “Greatly attenuated reactivity of nitrile-derived carbon-centered radicals toward oxygen”, Chem. Commun., 1576-1577; Font-Sanchis, E. et al. “Generation and Reactivity toward Oxygen of Carbon-Centered Radicals Containing Indane, Indene, and Fluorenyl Moieties” J. Org. Chem. (2003), 68, 3199-3204; Font-Sanchis, E. et al. “Reactivity toward Oxygen of Isobenzofuranyl Radicals: Effect of Nitro Group Substitution” Organic Letters, (2003), 5 (9), 1515-1518; and Aliaga, C. “A New Method to Study Antioxidant Capability: Hydrogen Transfer from Phenols to a Prefluorescent Nitroxide” Organic Letters (2003), 5 (22), 4145-4148, which are incorporated herein by reference.

Thermo-oxidative degradation can simplistically be described as a two-cycle process. Two different types of reaction work together to create organic radicals by hydrogen abstraction, and to activate molecular oxygen by reaction with the organic radicals, resulting in formation of peroxyl radicals, which in turn act as hydrogen abstraction active species to produce peroxides and organic radicals. Peroxides themselves serve as a source of hydrogen abstraction radicals by homolytic cleavage of the peroxo O—O bond. Different classes of antioxidants exist, which are characterized as primary antioxidants, secondary antioxidants, and carbon-centered radical scavengers. Primary antioxidants mainly act as chain-breaking antioxidants, reacting rapidly with peroxyl radicals. Secondary antioxidants react with peroxo compounds to yield non-radical, non-reactive products. Carbon-centered radical scavengers are very effective in trapping alkyl radicals, and provide powerful processing stability. Typical examples are carbon-centered radicals derived from benzofuranone derivatives.

When using antioxidants whose action is mediated by carbon-centered radicals, suitable precursors are typically employed, which after activation yield the carbon-centered radical. Often, such precursors are designed such that the radical is formed in a process of hydrogen abstraction. This follows a hydrogen transfer process.

There is a continuing need to develop novel antioxidant compounds and methods that enable rapid availability of antioxidant species. In particular, there is a need to develop compounds and methods that allow for modulation of antioxidant levels in a chosen system.

### SUMMARY OF THE INVENTION

It is an object of the present invention, at least in preferred embodiments, to provide a compound that can be “activated” to exhibit antioxidant activities.

It is another object of the present invention, at least in preferred embodiments, to provide a method for selective modulation of antioxidant levels in a reaction mixture or other system.

It is another object of the present invention, at least in preferred embodiments, to provide precursors of carbon-centered radicals that are ‘self-activating’ in response to a change in temperature.

It is another object of the present invention, at least in preferred embodiments, to provide precursors of carbon-centered radicals that are self-activating within a predetermined range of temperature values.

It is another object of the present invention, at least in preferred embodiments, to provide precursors of carbon-centered radicals in which the self-activating process is reversible.

The inventor has established that the mode of operation for specific types of antioxidants, involves the formation of carbon-centered radicals that do not react with oxygen. The inventor has further determined that/this mode of operation is most unusual since the vast majority of carbon-centered radicals react with oxygen at rates that approach diffusion control. Following up on this observation, the inventor has discovered a wide range of compounds that have the unusual property of producing carbon centered radicals that either do not react with oxygen, or where the reactivity is greatly attenuated.

The inventor's observations have led to the identification of an entirely new class of antioxidant compounds and activatable precursors that can be manipulated according to the ambient temperature conditions. Importantly, these systems present unique opportunities to provide reaction systems in which antioxidant levels are modulated by simple temperature shifts. Such systems may include, but are not limited to, plastics, lubricants, cooling fluids, and pharmaceutical/medical applications.

The antioxidant precursor compound of the present invention comprises a disassociatable dimerized compound having the formula A-B. The compound preferably comprises two monomers, A and B, connected by a carbon-carbon labile bond which is susceptible to breakage upon exposure to heat to form two carbon centered radicals A• and B•. These carbon centered radicals may act as antioxidants by scavenging or trapping oxidizing species, such as for example, carbon centered, peroxyl radicals or nitroxides. Such scavenging or trapping will lead to a disruption of unwanted oxidation processes thereby preventing or minimizing oxidation.

In one aspect of the invention there is provided a thermally activatable antioxidant precursor compound of the formula:

A-B
wherein A and B are the same or different, each consisting of a moiety other than a hydrogen atom; and
wherein A and B are connected via a labile bond, and are able to dissociate through breakage of the labile bond upon exposure of said compound to a predetermined temperature shift from a lower temperature to a higher temperature, thereby to generate corresponding free radicals A• and B• at least one of which being suitable for use as an antioxidant. Preferably, each of A and B comprises a monocyclic aromatic or polycyclic aromatic ring system, optionally substituted at one or more positions. Preferably, the labile bond is a carbon-carbon bond and each of the free radicals A• and B• is a carbon centered free radical. Preferably, the compound A-B is a dimer and each of the free radicals A• and B• is a monomer. Preferably, each of free radicals A• and B• are suitable for use as an antioxidant. Preferably, each of A and B further comprise a heterocyclic ring. Preferably, the free radicals A• and B• are able to re-associate through the formation of a labile bond upon exposure of said radicals to a predetermined temperature shift from a higher temperature to a lower temperature thereby to regenerate the corresponding antioxidant precursor compound of the formula:

A-B

More preferably, the compound A-B is a dimer and each of the free radicals A• and B• are monomers. Preferably, A and B are identical.

In another aspect, the compound A-B of the present invention is selected from compounds of the formula X:

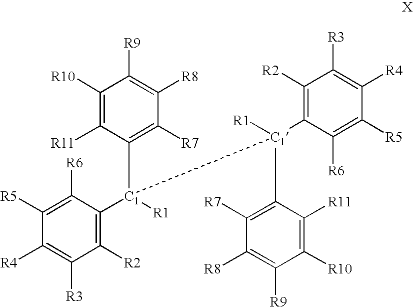

wherein the dashed line represents the labile bond susceptible to breakage upon exposure of said compound to a temperature shift from a lower temperature to a higher temperature; and
wherein R1-R11 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl,;
and wherein optionally any two of R2 to R11 within the same moiety of A or B may be linked to form a substituted or unsubstituted bicyclic or polycyclic fused ring system, and wherein optionally R1 may be linked to one or more of R2, R6, R7, and R11 within the same moiety of A or B, to form a substituted or unsubstituted bicyclic or polycyclic fused ring system optionally comprising one or more heterocyclic rings.

In another aspect, the compound of the formula A-B has a structure of formula I:

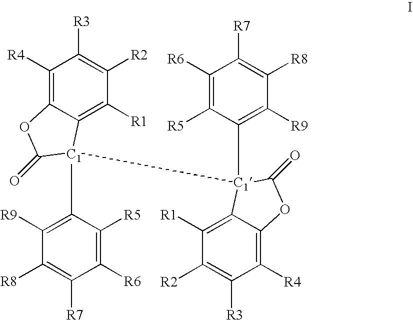

wherein the dashed line represents the labile bond susceptible to breakage upon exposure of said compound to a temperature shift from a lower temperature to a higher temperature; and
wherein R1-R9 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl.

In another aspect, the compound of the formula A-B has a structure of formula II:

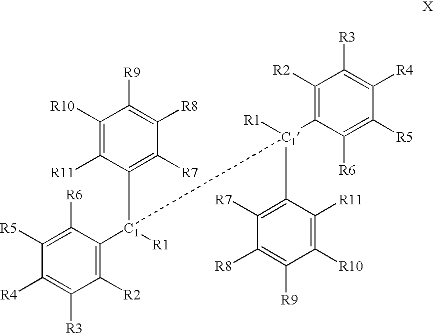

wherein the dashed line represents the labile bond susceptible to breakage upon exposure to a higher temperature; and
wherein R1represents an electron withdrawing group;
wherein R2-R11 are the same or different, each independently selected from hydrogen or a substituent selected from the following group: linear or branched C₁-C18 alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C2-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl. Preferably, the electron withdrawing group is selected from CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), and NO₂. Preferred electron withdrawing groups for R1 include CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), and NO₂.

In another aspect, the compound of the formula A-B has a structure of formula III:

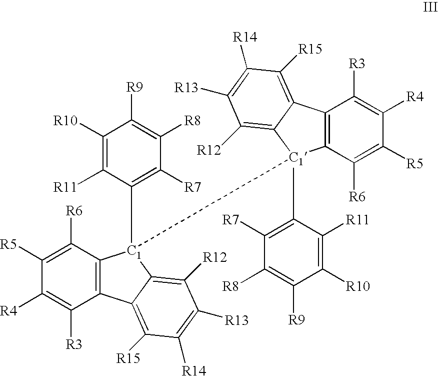

wherein the dashed line represents the labile bond susceptible to breakage upon exposure to a higher temperature; and
wherein R3-R15 are the same or different, each independently selected from hydrogen or a substituent selected from the following group: linear or branched C₁-C₁₈ alkyl, linear or branched C2-C₁₈ alkenyl, linear or branched C₂-C₁₈ alkynyl, CN, CHal3 (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl.

In another aspect, the compound of the formula A-B has a structure of formula IV:

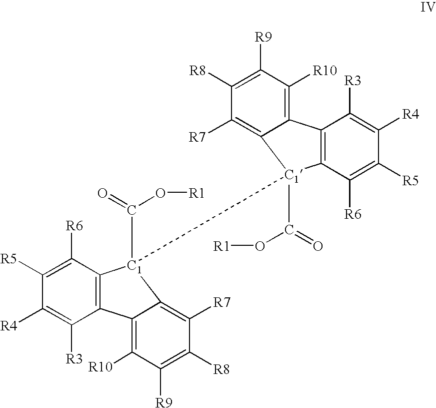

wherein the dashed line represents the labile bond susceptible to breakage upon exposure to a higher temperature; and
wherein R1 and R3 to R10 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl.

In another aspect, the compound of the formula-A-B has a structure corresponding to formula IA:

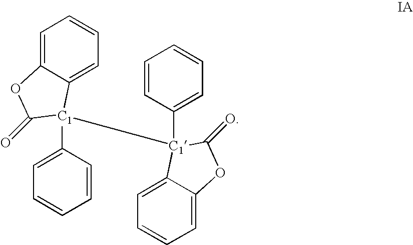

In another aspect, the compound of the formula A-B has a structure corresponding to formula IB:

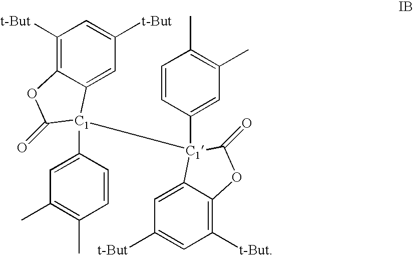

In another aspect, the compound of-the formula A-B has a structure corresponding to formula IIA:

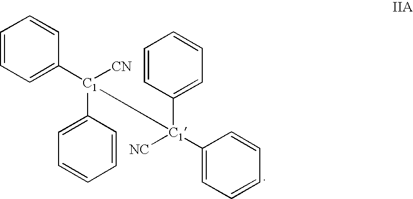

In another aspect, the compound of the formula A-B has a structure corresponding to formula IIIA:

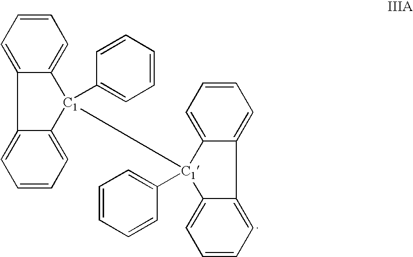

In another aspect, the compound of the formula A-B has a structure corresponding to formula IVA:

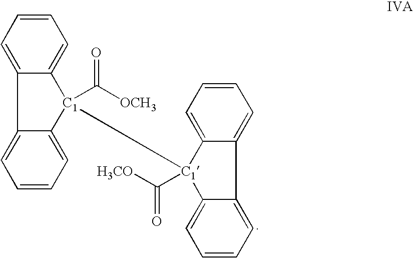

In another aspect of the compounds of the invention, one of or both of the free radicals A• and B• are of the formula V:

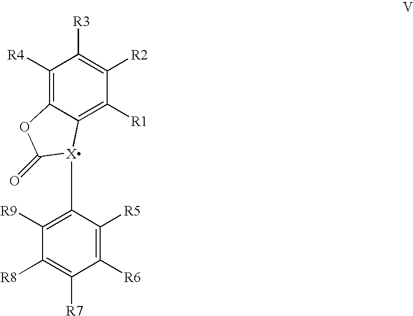

wherein X represents C₁ or C₁′ and R1 to R9 are the same or different, each representing hydrogen or a substituent selected from the following group: linear C₁-C₁₈ alkyl, branched C₁-C₁₈ alkyl, linear C₂-C₁₈ alkenyl, branched C₂-C₁₈ alkenyl, linear C₂-C₁₈ alkynyl, branched C₂-C₁₈ alkynyl, C₅-C₈ cycloalkyl optionally substituted with C₁-C₁₈ alkyl groups, and C₆-C₂₀ aryl optionally substituted with C₁-C₁₈ alkyl groups.

In another aspect of the compounds of the invention, one of or both of the free radicals A• and B• are of the formula VI:

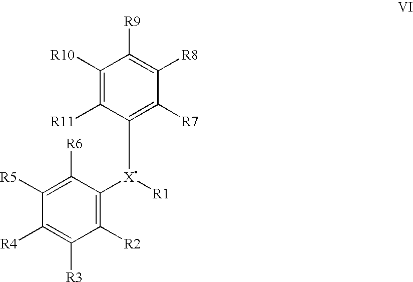

wherein X represents C₁ or C₁′ and R1 represents an electron withdrawing group, most preferably selected from, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), and NO₂;
and R2-R11 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ allyl.

In another aspect of the compounds of the invention, one of or both of the free radicals A• and B• are of the formula VII:

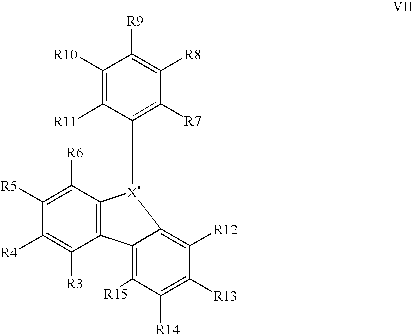

wherein X represents C₁ or C₁′ and R3-R15 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C]₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl.

In another aspect of the compounds of the invention, one of or both of the free radicals A• and B• are of the formula VIII:

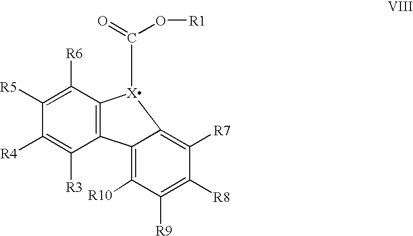

wherein X represents C₁ or C₁′ and R1 and R3 to R10 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl.

In another aspect of the compounds of the invention, one or both of the free radicals A• and B• are of the formula VA:

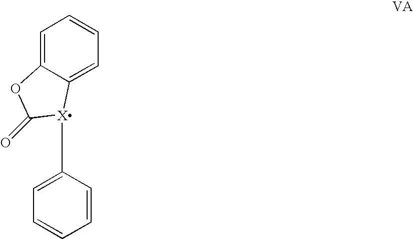

wherein X represents C₁ or C₁′.

In another aspect of the compounds of the invention, one or both of the free radicals A• and B• are of the formula VB:

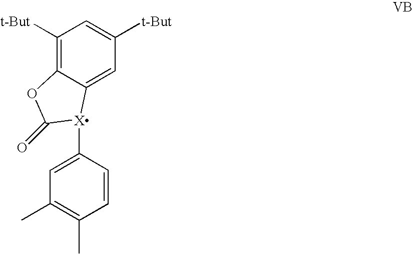

wherein X represents C₁ or C₁′.

In another aspect of the compounds of the invention, one or both of the free radicals A• and B• are of the formula VIA:

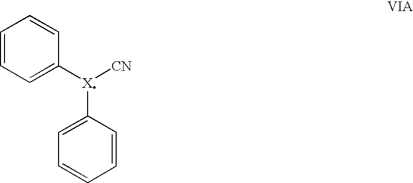

wherein X represents C₁ or C₁′.

In another aspect of the compounds of the invention, one or both of the free radicals A• and B• are of the formula VIIA:

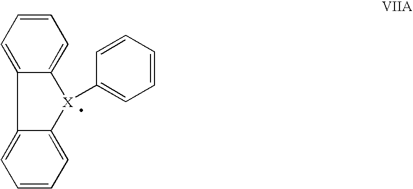

wherein X represents C₁ or C₁′.

In another aspect of the compounds of the invention, one or both of the free radicals A• and B• are of the formula VIIIA:

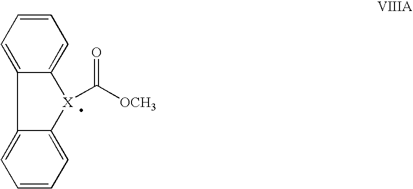

wherein X represents C₁ or C₁′.

In another aspect of the compounds of the invention, one or both of A and B is selected from the group consisting of:

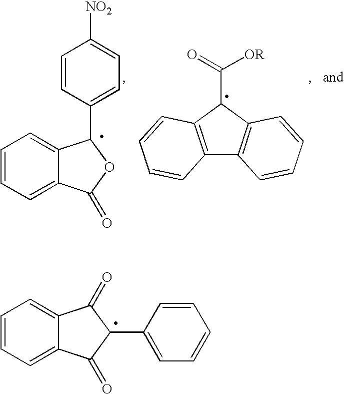

wherein R is selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C8 cycloalkyl optionally substituted with one or more C₁-C₁₈ alkyl, and C₆-C₂₀ aryl, optionally substituted with one or more C₁-C₁₈ alkyl.

In another aspect of the compounds of the invention, one of or both of the free radicals A• and B• are of the formula IX:

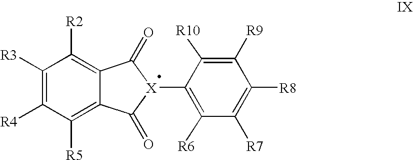

wherein X represents C₁ or C₁′, and R2 to R10 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally

In another aspect, the present invention provides compounds of the formula A-B as described herein, with the proviso that the compound is not the compound of formula (11) disclosed in U.S. Pat. No. 5,367,008 or the compound of formula (10) disclosed in U.S. Pat. No. 5,428,177 or the dimeric form of Irganox HP-136 (Ciba Speciality Products) or related dimeric products.

Preferably, in the compounds A-B of the present invention, the bond dissociation energy of the labile bond is less than 80 kcal/mol. More preferably, the bond dissociation energy of the labile bond is less than 50 kcal/mol. More preferably, the bond dissociation energy of the labile bond is less than 25 kcal/mol. More preferably, the bond dissociation energy of the labile bond is less than 20 kcal/mol.

Preferably, in the thermally activatable antioxidant compound of the present invention, said temperature shift is from a lower temperature of from 0° C. to 40° C. to a higher temperature of from 20° C. to 40° C.

In another aspect the present invention provides for the use of a compound of the formula:

A-B
as defined herein, as a thermally activatable antioxidant precursor compound. In another aspect the present invention provides for a composition comprising: (a) a compound susceptible to oxidation; and (b) the thermally activatable antioxidant precursor compound as described herein. Preferably, the compound (a) is more susceptible to oxidation at a higher temperature than at a lower temperature.

In another aspect the present invention provides for a method for generating an antioxidant, the method comprising the steps of:

a) providing an antioxidant precursor compound of the formula:

A-B
as described herein; and

b) adjusting the temperature of A-B to thereby cause dissociation of the compound into free radicals A• and B•. Preferably, the antioxidant precursor compound is substantially dormant prior to the step b) of adjusting. Preferably, the antioxidant precursor compound can be at least in part reformed at a lower temperature; step b) comprising heating the antioxidant precursor compound to a higher temperature, the method further comprising step c) cooling the free radicals A• and B• formed in step b) to a lower temperature to thereby cause at least partial reassociation of the free radicals A• and B• into the thermally activatable antioxidant precursor compound A-B.

Preferably, the step of adjusting comprises shifting the temperature of the antioxidant precursor compound from a lower temperature of from 0° C. to 40° C. to a higher temperature of from 20° C. to 40° C.

Preferably, the free radicals A• and B• are monomers and reassociation comprises dimerization of the free radicals A• and B• to regenerate A-B.

In another aspect the present invention provides for a method of preventing or slowing oxidation of at least one molecule susceptible to oxidation in a reaction mixture or target environment, the method comprising the steps of:

a) providing an antioxidant precursor compound of the formula:

A-B
as described herein;

b) adding the compound to the reaction mixture or target environment; and

c) if necessary adjusting a temperature of the reaction mixture or target environment to a temperature sufficient to cause dissociation of the compound into free radicals A• and B•.

Preferably, the step of adjusting comprises shifting the temperature of the antioxidant precursor compound from a lower temperature of from 0° C. to 40° C. to a higher temperature of from 20° C. to 400° C.

Preferably, the temperature sufficient to cause dissociation of the compound into free radicals A• and B• is a higher temperature portion of a thermal cycle. More preferably, the antioxidant precursor compound dissociates into the free radicals A• and B• at a temperature lower than a desired temperature for said reaction mixture or target environment.

Preferably, the thermal cycle includes a lower temperature portion following the higher temperature portion, the method further comprising step d) cooling the reaction mixture or target environment to the lower temperature portion of the thermal cycle thereby to cause at least partial reassociation of A and B to form the thermally activated antioxidant precursor compound A-B.

Preferably, the compound A-B is substantially dormant at the lower temperature portion of the thermal cycle.

In another aspect the present invention provides a composition comprising at least one molecule susceptible to oxidation, and two or more compounds A-B according to the present invention, each of said two or more compounds having alternative combinations of moieties A and B. Preferably, each of said two or more compounds comprises a labile bond having a bond strength that is different from all other compounds of said two or more compounds. Preferably, said composition is suitable for subjection to a thermal cycle to cause selective dissociation of moieties A and B for each of said two or more compounds.

In another aspect, the present invention provides for a method for synthesizing the thermally activatable antioxidant precursor compound A-B of the present invention, the method comprising the steps of:

a) providing a mixture comprising A-H, B—H and tert-butyl peroxide, wherein each H is a hydrogen atom; and

b) performing a photolysis reaction to produce t-BuOH and A-B.
Preferably, the moieties A and B are identical.
Preferably, photolysis is carried out at 350 nm.

### BRIEF DESCRIPTION OF THE DRAWINGS

FIG. 1A shows a graph which illustrates the lifetime of a benzyl radical at various temperatures in a control experimental environment lacking the thermally activatable antioxidant compounds of the present invention. Not that the changes in temperature cause only small changes in the-benzyl radical decay.

FIG. 1B shows a graph which illustrates the lifetime of a benzyl radical at various temperatures when in the presence of a diphenylacetonitrile antioxidant precursor compound of the present invention, whereby the temperature shifts cause more significant changes in the benzyl radical decay.

FIG. 2 shows the absorption spectra of the monomer-radical obtained by thermal dissociation of compound IB at different temperatures.

FIG. 3A shows a Van't Hoff plot for some of the thermally activatable antioxidant precursor compounds of the present invention wherein the vertical axis is proportional to the concentration of the corresponding antioxidant radical at a given temperature. The plot represented by □ illustrates the behaviour of 9-Ph-fluorene, the plot represented by ⋄ illustrates the behaviour of HP-136 dimer, the plot represented by ∘ illustrates the behaviour of Ph-cumaranone, and the plot represented by Δ illustrates the behaviour of Ph₂CHCN.

FIG. 3B provides a further Van't Hoff plot, 336 nm, 48° C. to 102° C., 17.36 mM Diphenyl Acetonitrile under nitrogen.

FIG. 4 shows a crystal structure of a thermally activatable antioxidant precursor compound derived from radical monomer HP-136, whereby the bond between C48 and C24 represents the labile bond of the thermally activatable antioxidant precursor compound of the present invention.

FIG. 5 provides a flow diagram of a method of providing oxidation protection according to an embodiment of the instant invention.

FIG. 6 provides a flow diagram of a method of providing oxidation protection according to another embodiment of the instant invention.

FIG. 7 provides a flow diagram of a method for generating antioxidant molecules in accordance with one embodiment of the invention.

FIG. 8 provides a flow diagram of a method of preventing or slowing oxidation in accordance with one embodiment of the present invention.

FIG. 9 provides a flow diagram of a method of synthesizing a thermally activatable antioxidant compound according to one embodiment of the present invention.

FIG. 10 shows a graph illustrating the percentage degree of HP-136 (dimer) dissociation at 0.1% loading (y-axis) plotted against temperature (x-axis)

FIG. 11 is a graph equivalent to FIG. 10 with the exception that the y-axis has a log scale.

### DEFINITIONS

Activated: The term “activated” applies generally to the state whereby dissociation of the thermally activatable antioxidant precursor compounds of the present invention has increased relative to a dormant state thereby to cause the generation of at least one free radical antioxidant.
Dimer: It is to be understood that throughout the description and in the claims that follow, the term “dimer” refers to compounds comprising two major moieties or monomers connected via a covalent labile bond. Each dimer may dissociate via breakage of the labile bond to form two monomeric free radicals, each comprising one of the major moieties of the dimer. It will further be understood that the term ‘dimer’ preferably, but not necessarily, refers to compounds wherein the two major moieties or monomers are identical.
Dormant: For the purposes of this specification dormant refers to there being little to no activity of the antioxidant precursor compounds with respect to the dissociation of the precursor compounds into the corresponding carbon centered free radical antioxidants. However, some dissociation may occur even in a dormant state.
Higher Temperature: It is to be understood that throughout the description and the claims that follow, the term “higher temperature” is used relative to a “lower temperature” whereby the antioxidant precursor compounds show a higher antioxidant activity at a higher temperature relative to the lower temperature due to increased dissociation into active antioxidant radicals. In contrast, at lower temperatures the antioxidant precursor compounds are substantially dormant (see definition of dormant). At a preferred higher temperature, the equilibrium shifts towards dimer dissociation into the respective monomers, which exhibit activity as antioxidant radicals. Even at higher temperatures, there may only be a very small amount of disassociated molecules compared to undissociated molecules. At higher temperatures there is at least a slight shift in the equilibrium of the dimer, thereby causing at least a small amount of dissociation of the dimer into the corresponding free radical monomers. The precise value of a higher temperature will vary depending upon the moieties/monomeric units used to make up the antioxidant precursor compounds and it will be appreciated that the bond dissociation energy of the carbon-carbon labile bond will dictate suitable lower and higher temperatures to cause at least some dissociation of the antioxidant precursor compounds into the corresponding antioxidant free radicals. In preferred embodiments, a higher temperature will comprise a temperature of at least 20° C., more preferably at least 25° C., more preferably at least 30° C., more preferably at least 40° C. Preferably, a higher temperature will not be more than 400° C.
Labile bond: A carbon-carbon bond having a bond strength that is less than an average carbon-carbon sp³ bond strength (i.e., typically, for example, less than 80-90 kcal/mol). The antioxidant precursor compounds of the present invention include a labile bond between moieties/monomeric units of each dimer. The labile bond is broken when the antioxidant precursors are activated to form two carbon centered free radicals. Reassociation or regeneration of the antioxidant precursors is done through the reformation of the labile bond between two suitable carbon centered radicals, which may in part be induced by a shift in temperature from a higher temperature to a lower temperature.
Lower Temperature: It is to be understood that throughout the description and the claims that follow, the term “lower temperature” is used with reference to a “higher temperature” whereby the antioxidant precursor compounds show a lower activity at a lower temperature relative to the higher temperature whereby the antioxidant precursor compounds are at least in part activated. At preferred lower temperatures, the equilibrium shifts towards dimerization of respective monomers to generate the antioxidant precursor compounds. However, even at lower temperatures, there may be some dissociation of the dimer in to the respective monomers. At lower temperatures there must be at least a slight shift in the equilibrium towards, dimerization relative the equilibrium at higher temperatures. The lower temperature will vary depending upon the moieties/monomeric units used to make up the antioxidant precursor compounds and it will be appreciated that the bond dissociation energy of the carbon-carbon labile bond will dictate suitable lower temperatures to slow dissociation of the antioxidant precursor compounds into the corresponding antioxidant free radicals. In preferred embodiments, a lower temperature comprises a temperature less than 40° C., more preferably less than 30° C., more preferably less than 20° C. Preferably, a lower temperature is more than 0° C.
Monomer: It is to be understood that throughout the description and in the claims that follow, the term “monomer” preferably refers to one or two moieties of a dimer linked via a labile bond, or alternatively refers to a carbon centered radical moiety generated by breakage of a labile bond of a dimer. When making up a dimer, the two monomers may have identical structures or may have different structures, the two monomers linked via a carbon-carbon labile bond. “Identical” monomers have matching structures and include isomers, stereo isomers and mirror image isomers.
Oxidation Temperature: Temperature at which a molecule has at least some degree of susceptibility to oxidation. Preferably, at the oxidation temperature, an increased susceptibility to oxidation is observed.
Preferably: the term ‘preferably’ precedes mention of a preferred feature of the invention. Unless stated otherwise, the preferred feature described pertains to a preferred feature of the broadest embodiments of the invention.
Substituent: encompasses any group that may reasonably replace hydrogen in a compound of the present invention. Particularly preferred substituents are selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈-cycloalkyl optionally substituted with one or more C₁-C₁₈ alkyl, and C₆-C₂₀ aryl, optionally substituted with one or more C₁-C₁₈ alkyl. In preferred embodiments, substituents may be linked to form a substituted or unsubstituted bicyclic or polycyclic fused ring system, such that any substituents to such a ring system may be selected from the list defined above.
Temperature Shift/adjustment of a temperature: A shift from one temperature to another. Preferably, such a shift in temperature refers to a change in temperature between either the lower, higher or oxidizing temperature (as defined herein) thereby causing a change in the equilibrium of a thermally activatable antioxidant precursor compound of the invention, to increase or decrease the degree of dissociation of the moieties of the compound into free radicals. Such a shift in temperature may take any form and occur by any reason. In specific embodiments of the invention, a temperature shift may involve an active and intentional change in the thermal environment of the compound of the invention, for example by actively adjusting operating conditions of the system to which the compound is being applied. For example, the compound of the invention may be employed in a chemical reaction system with a precisely controlled thermal cycle. In alternative specific embodiments, such a temperature shift may arise from inherent characteristics, features, or operating conditions of a system or apparatus to which the compound of the invention is being applied. For example, the compound of the invention may be employed as an additive to an enclosed oil system for use in connection with an internal combustion engine, whereby a temperature shift may be an inevitable result, and an inherent characteristic of the engine under normal operating conditions.
Target environment: For the purposes of this specification, refers to any suitable environment within which a thermal cycle may be performed or in which dissociation of an antioxidant precursor compound may be carried out.
Thermally activatable antioxidant precursor: For the purposes of this specification, any compound having the general structure A-B wherein A and B are the same or different, each of A and B comprise a monocyclic aromatic or polycyclic aromatic ring system, optionally substituted at one or more positions, including substitution by a heterocyclic ring;
and wherein A and B are connected via a labile bond, and are susceptible to dissociation through breakage of the labile bond upon exposure of the compound to a predetermined temperature shift from a lower temperature to a higher temperature, thereby to generate corresponding free radicals A• and B• at least one of which being suitable for use as an antioxidant.
Thermally activated antioxidant: The free radical generated from the thermal activation of the thermally activatable antioxidant precursor having at least some antioxidizing capability. Thermal activation causes dissociation of the carbon-carbon labile bond between the moieties/monomeric units of the thermally activatable antioxidant precursor thereby preferably resulting in at least one carbon centered free radical having at least some antioxidizing capability.

### DETAILED DESCRIPTION OF THE PREFERRED EMBODIMENTS

In principle, stable or persistent free radicals can act as antioxidants, i.e., a stable free radical that is either capable of trapping or scavenging carbon centered or peroxyl radicals may act as an antioxidant, as such trapping will lead to a disruption of the oxidation chain. In fact, stable radicals or their precursors (HALS) are well established polymer additives.

The inventors have succeeded in developing antioxidant precursor compounds which, upon exposure to heat, undergo at least partial dissociation into free radicals which preferably have antioxidant characteristics.

The antioxidant precursor compounds have a dimerized scaffold of the formula A-B. The scaffold is preferably comprised of two moieties/monomeric units connected via a carbon-carbon labile bond which is broken or disassociates upon exposure to heat, thus forming carbon centered free radicals A• and B•, as seen in reaction scheme 1. At least one of the free radicals A• and B• behave as an antioxidant. However, in preferred embodiments of the present invention, both free radicals A• and B• behave as antioxidants thus resulting in a 2:1 ratio of antioxidants generated by dissociation of the precursor compound.

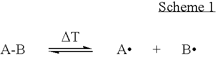

In one aspect, the present invention pertains, at least in preferred embodiments, to compounds that are not themselves good antioxidants, since they are neither free radicals, nor do they have any labile hydrogen atoms. Because of their very low bond dissociation energy (normal C—C bond energies are much higher, typically 80-90 kcal/mol) these compounds dissociate at moderately high temperatures producing a large concentration of free radicals that are themselves capable of scavenging the radicals that mediate the oxidative degradation of materials. These compounds may be regarded as “dormant” antioxidants or antioxidant precursor compounds. Their antioxidant activities are activated by heating. Some examples of the compounds of the present invention produce a low radical concentration, for example at 40° C., but the radical concentration is greatly enhanced at for example 100° C. or higher. It is possible to achieve large concentrations of antioxidant radicals, with 10 percent or more of the dimeric compound being dissociated into active antioxidant (monomeric) form.

An important preferred characteristic of the compounds of the present invention is the reversibility of the dimer to monomer transition. Thus, if the antioxidant free radicals A• and B• are cooled, the dimeric compound or the antioxidant precursor compound is at least partial regenerated by reassociation of carbon-carbon labile bonds. This reassociation includes the formation of a carbon-carbon labile bond between monomeric units or moieties which were previously not bonded to each other. The precursor compound may reform and again exhibit dormancy in terms of antioxidant properties. Most preferably, the compounds of the present invention may be subjected to multiple thermal cycles between a lower temperature and a higher temperature thereby cycling the precursor compound between inactive (dimeric) and active antioxidant (monomeric free radical) forms.

The carbon centered free radicals At and Be of the present invention act as antioxidants by scavenging or trapping various oxidizing species including carbon or peroxyl centered free radicals, thus interrupting the oxidation chain. An example of such interruption is shown in reaction scheme 2. Reaction scheme 2 shows an exemplary break down by light of a typical polymer, for example dibenzyl ketone, into the corresponding carbon centered benzyl radical. The carbon centered benzyl radical is prone to oxidation thereby resulting in the breakdown of the corresponding compound. Reaction scheme 3 shows the activation or dissociation of the antioxidant precursors of the present invention by exposure to heat, for example, of the diphenylacetonitrile dimer, into the corresponding free radical antioxidant monomers. The heat provides enough energy to overcome the bond dissociation energy of the labile carbon-carbon bond thus breaking the antioxidant precursor into the corresponding monomers or antioxidant free radicals. The antioxidant free radicals may be used to trap or scavenge the carbon centered benzyl radical as shown in reaction scheme 4, or to trap or scavenge oxygen centered radicals as shown in reaction scheme 5.

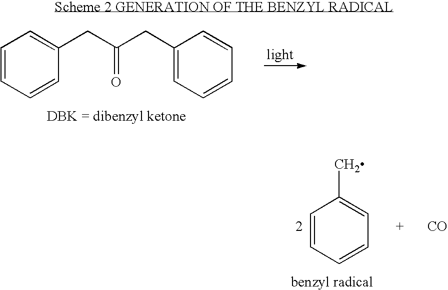

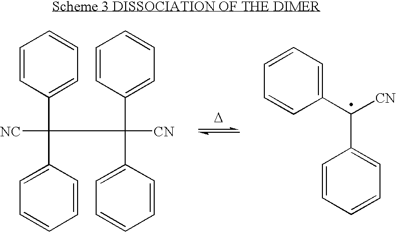

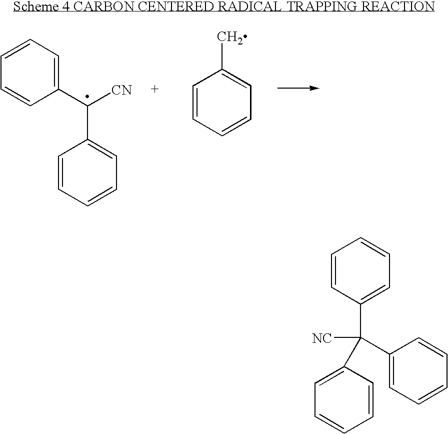

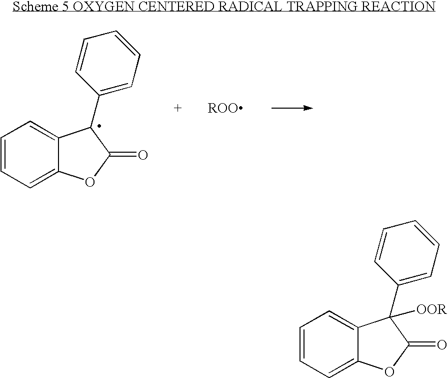

When the antioxidant precursor compound(s) of the present invention is present in the reaction mixture or target environment and the temperature increase, it has been shown that the lifetime of the benzyl radicals decrease dramatically. This demonstrates that the antioxidant precursor, upon “activation”, produces radicals capable of scavenging or trapping other radicals, in this case the benzyl radical. The reactions may be carried out in air.

In preferred embodiments, the compound A-B is selected from compounds of the formula X:

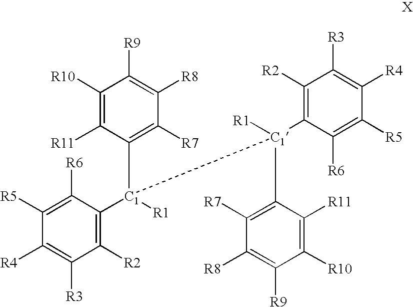

wherein the dashed line represents the labile bond susceptible to breakage upon exposure of said compound to a temperature shift from a lower temperature to a higher temperature; and
wherein R1-R11 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl;
and wherein optionally any two of R2 to R11 within the same moiety of A or B may be linked to form a substituted or unsubstituted bicyclic or polycyclic fused ring system, and wherein optionally R1 may be linked to one or more of R2, R6, R7, and R11 within the same moiety of A or B, to form a substituted or unsubstituted bicyclic or polycyclic fused ring system optionally comprising one or more heterocyclic rings. The inventors have extensive evidence that various compounds falling within this group of compounds are competent thermally activatable antioxidant precursor compounds.

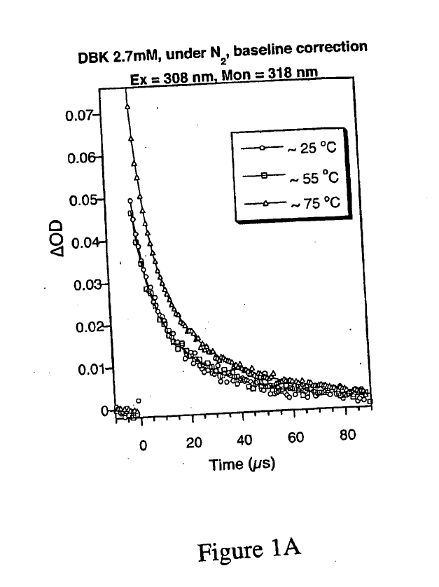

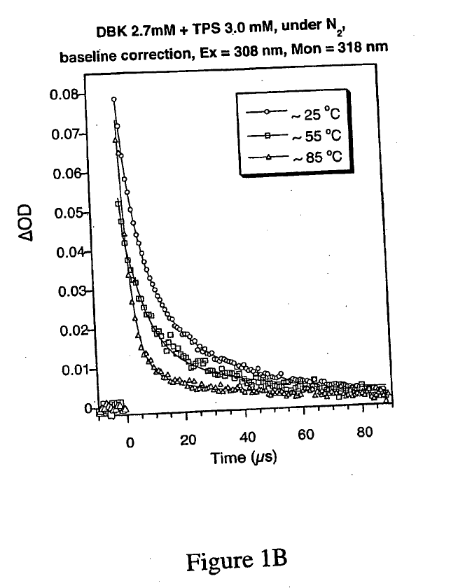

Shown in FIG. 1A is a graph illustrating the lifetime of dibenzyl ketone radical decay at various temperatures as measured by absorption characteristics. FIG. 1A shows the rate of decay in the absence of antioxidant precursor compounds or the corresponding free radical antioxidants. It is observed that as temperature increases, the rate of decay of the benzyl radical slightly decreases. FIG. 1B, shows a similar dibenzyl ketone radical decay. However, in contrast to FIG. 1A, the reaction mixture included an antioxidant precursor (TPS) of the present invention. It is observed that as temperature increases, and the precursors are activated, the rate of decay of the benzyl radical is significantly increased. The antioxidant free radicals of the present invention therefore presumably trap or scavenge the benzyl radicals thus interrupting the oxidation chain.

In one aspect, the present invention pertains to a wide range of antioxidant precursor compounds each comprising a labile C—C bond. The nature of the moieties adjacent the labile bond effectively determines the bond dissociation energy (BDE) of the labile bond. Therefore, the present invention pertains to a range of compounds with a range of BDE values, such that each compound dissociates into corresponding monomer radicals at a different predetermined temperature. In this way, a compound of the invention may be selected and tailored to a specific process or use in accordance with its expected dissociation temperature or temperature range.

According to selected embodiments of the present invention, dimeric compounds are utilized as thermally activatable dormant antioxidant precursors. Presented below are some examples of antioxidant precursor compounds of the present invention having an A-B structure connected via a labile carbon-carbon bond between C₁ and C₁′ indicated by a dashed line. An example of the thermally activatable antioxidant precursor compounds of the present invention are the compounds of formula I:

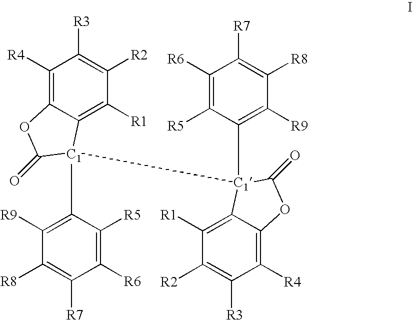

Preferred compounds of formula I are those wherein R1 to R9 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl. Further preferred compounds of formula I are those wherein the C₅-C₈ cycloalkyl groups carry C₁-C₁₈ alkyl groups as substituents. Further preferred compounds of formula I are those wherein the C₆-C₂₀ aryl groups carry C₁-C₁₈ alkyl groups as substituents.

Another example of the thermally activatable antioxidant precursor compounds of the present invention are the compounds of formula II:

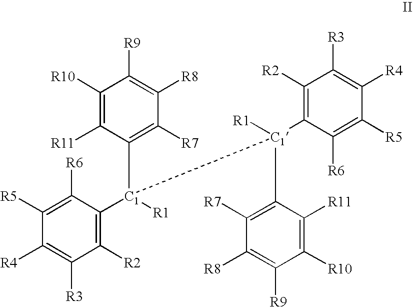

Preferred compounds of formula II are those wherein R1 represents an electron withdrawing group, and R2 to R11 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl. Preferably, R1 is selected from CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), and NO₂. Further preferred compounds of formula II are those wherein the C₅-C₈ cycloalkyl groups carry C₁-C₁₈ alkyl groups as substituents. Further preferred compounds of formula II are those wherein the C₆-C₂₀ aryl groups carry C₁-C₁₈ alkyl groups as substituents.

A third example of the thermally activatable antioxidant precursor compounds of the present invention are the compounds of formula III:

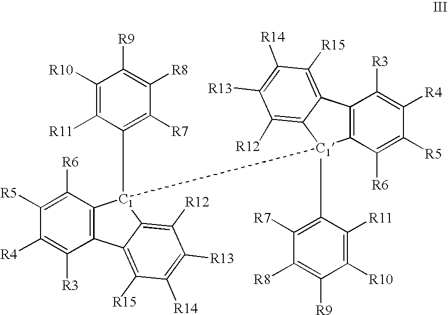

Preferred compounds of formula III are those wherein R3 to R15 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl.

A fourth example of the thermally activatable antioxidant precursor compounds of the present invention are the compounds of formula IV:

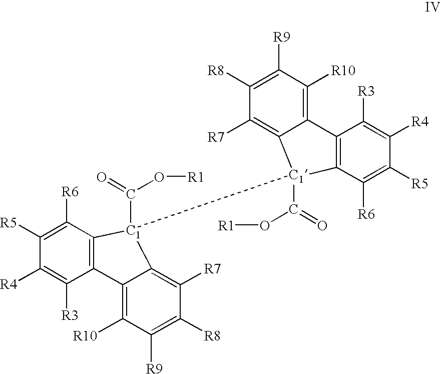

Preferred compounds of formula IV are those wherein R1 and R3 to R10 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl.

A preferred compound of formula I, in which R1, R2, R3, R4, R5, R6, R7, R8, and R9 are hydrogen, is illustrated by formula IA, below:

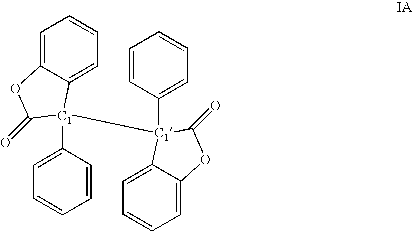

Another preferred compound of formula I, in which R1, R3, R5, R6, and R9 are hydrogen, R2 and R4 are tert-butyl and R7 and R8 are methyl, is illustrated by formula IB, below:

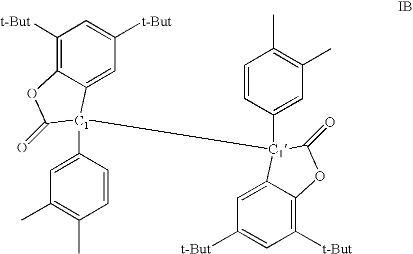

A preferred compound of formula II, in which R1 is a nitrile group, and R2, R3, R4, R5, R6, R7, R8, R9, R10, and R11 are hydrogen, is illustrated by formula IIA, below:

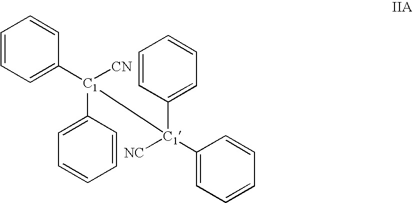

A preferred compound of formula III, in which R3, R4, R5, R6, R7, R8, R9, R10, R11, R12, R13, R16 and R15 are hydrogen, is illustrated by formula IIIA, below:

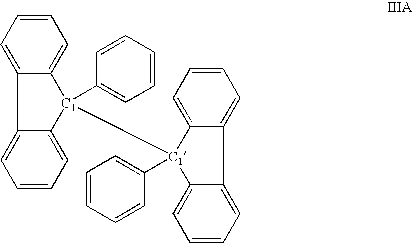

A preferred compound of formula IV, in which R1 is methyl and R3, R4, R5, R6, R7, R8, R9 and R10 are hydrogen, is illustrated by formula IVA, below:

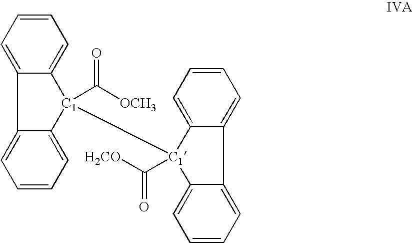

One of ordinary skill in the art will envisage other similar dimers that may be used with the methods according to embodiments of the invention.

The compounds of formula I, formula II, formula III, and formula IV are characterized by a weak C₁-C₁′ bond. Upon heating, at least a portion of the compound of formula I, formula II, formula III, or formula IV may undergo a C—C bond cleavage reaction, to produce a pair of radical-monomers, each one of the radical-monomers being a carbon-centered radical. It is the carbon-centered radicals, and not the compound itself, that principally function as an active antioxidant species. It should be noted that the antioxidant activity does not require or involve hydrogen transfer. Further, antioxidant radical generation is through dissociation of a dimeric compound and not through hydrogen transfer or abstraction, as required for many corresponding antioxidant compounds of the prior art. Preferably, the radical-monomers resulting from the dissociation of the dimeric compounds are stable carbon-centered radicals with greatly attenuated reactivity toward oxygen.

In one aspect, the present invention also pertains to the antioxidant free radicals obtained from thermal activation of the antioxidant precursor compounds having the formula A-B as described above. Exemplary antioxidant free radicals of the present invention are the free radicals corresponding to the precursor compounds of formula I, and are those of the formula V:

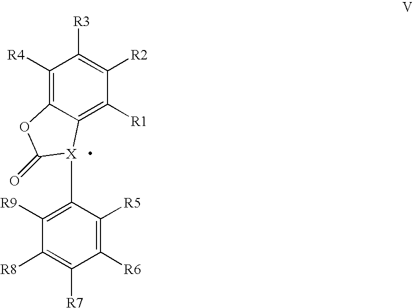

Preferred compounds of formula V are those wherein X represents C₁ or C₁′ and R1 to R9 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal3 (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl. Further preferred compounds of formula V are those wherein the C₅-C₈ cycloalkyl groups carry C₁-C₁₈ alkyl groups as substituents. Further preferred compounds of formula V are those wherein the C₆-C₂₀ aryl groups carry C₁-C₁₈ alkyl groups as substituents.

Other exemplary antioxidant free radicals of the present invention are the free radicals corresponding to the precursor compounds of formula II, and are those of the formula VI:

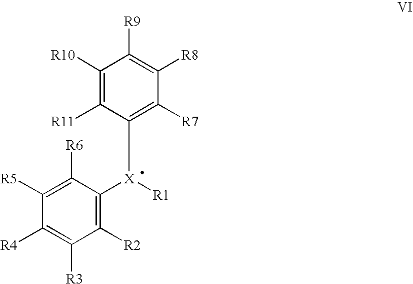

Preferred compounds of formula VI are those wherein X represents C₁ or C₁′ and R1 represents an electron withdrawing group, most preferably nitrile, and R2 to R11 are the same or different, each representing hydrogen or a substituent selected from the following non-limiting group: linear C₁-C₁₈ alkyl, branched C₁-C₁₈ alkyl, linear C₂-C₁₈ alkenyl, branched C₂-C₁₈ alkenyl, linear C₂-C₁₈ alkynyl, branched C₂-C₁₈ alkynyl, C₅-C₈ cycloalkyl, and C₆-C₂₀ aryl. Further preferred compounds of formula U are those wherein the C₅-C₈ cycloalkyl groups carry C₁-C₁₈ alkyl groups as substituents. Further preferred compounds of formula II are those wherein the C₆-C₂₀ aryl groups carry C₁-C₁₈ alkyl groups as substituents

Other exemplary antioxidant free radicals of the present invention are the free radicals corresponding to the precursor compounds of formula III, and are those of the formula VII:

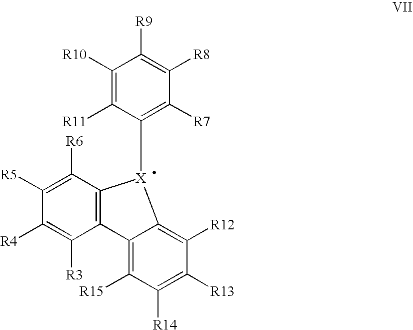

Preferred compounds of formula III are those wherein X represents C₁ or C₁′ and R3 to R15 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl.

Other exemplary antioxidant free-radicals of the present invention are the free radicals corresponding to the precursor compounds of formula IV, and are those of the formula VIII:

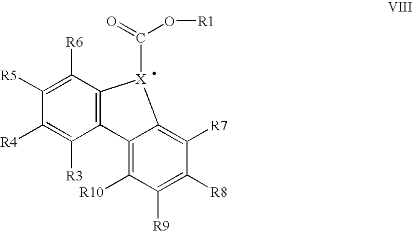

Preferred compounds of formula IV are those wherein X represents C₁ or C₁′ and R1 and R3 to R10 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl.

A preferred compound of formula V is that of the free radical corresponding to the thermally activatable antioxidant precursor compound of formulas IA, having the formula VA:

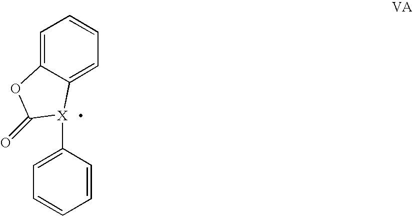

wherein X represents C₁ or C₁′.

Another preferred compound of formula V is that of the free radical corresponding to the thermally activatable antioxidant precursor compound of formulas IB, having the formula VB:

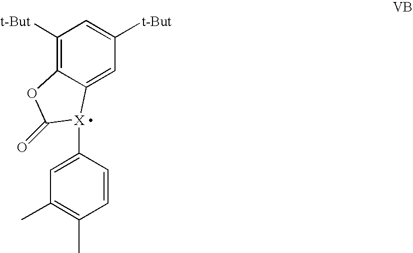

wherein X represents C₁ or C₁′.

A preferred compound of formula VI is that of the free radical corresponding to the thermally activatable antioxidant precursor compound of formulas IIA, having the formula VIA:

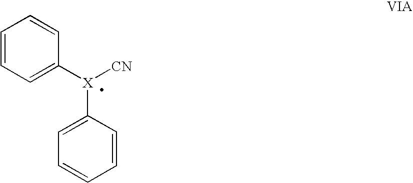

wherein X represents C₁ or C₁′.

A preferred compound of formula VII is that of the free radical corresponding to the thermally activatable antioxidant precursor compound of formulas IIIA, having the formula VIIA:

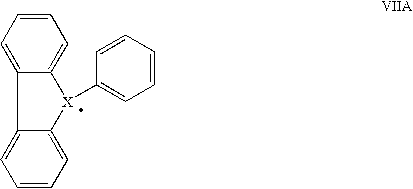

wherein X represents C₁ or C₁′.

A preferred compound of formula VII is that of the free radical corresponding to the thermally activatable antioxidant precursor compound of formulas IVA, having the formula VIIIA:

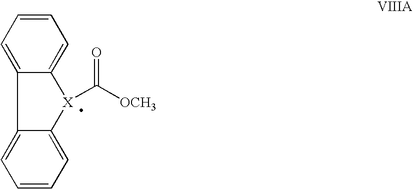

wherein X represents C₁ or C₁′.

The inventors have further succeeded in synthesizing carbon centered radicals having the following structures:

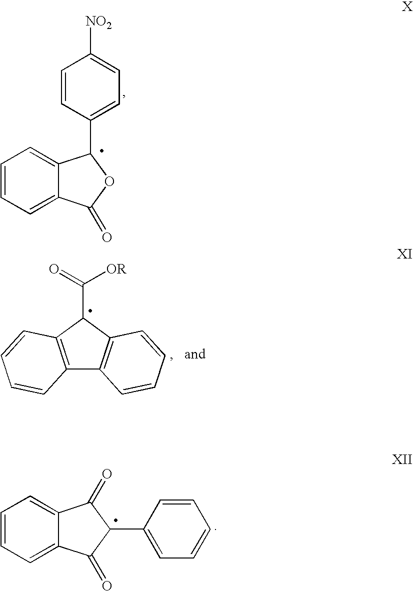

These radicals do not react with oxygen and it is expected that they will also exhibit some degree of antioxidant behaviour and/or dimerization.

Without wishing to be bound by theory, it is believed that the lack of reactivity toward oxygen of the antioxidant precursors and correspondingly generated free radicals of the present invention is attributable, at least in part, to several parameters, including but not limited to (a) benzylic resonance stabilization; (b) favourable stereoelectronic effects; (c) unpaired spin delocalization on heteroatoms and particularly oxygen; (d) electron withdrawing effects, and/or (e) steric effects.

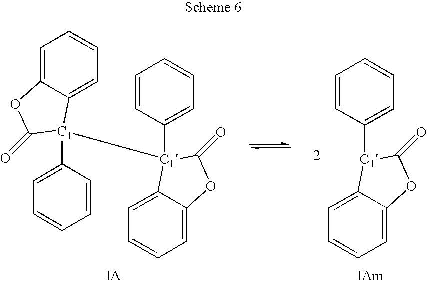

Reaction scheme 6 illustrates a C—C bond cleavage reaction for a specific and non-limiting example of compound IA, which reversibly dissociates to form two carbon-centered radical-monomers IAm. Thus, heating effectively “activates” the antioxidant properties of the dimeric compounds by formation of the corresponding radical monomers. For example, when in solution, heating a sample compound to a temperature of 40° C. may produce a small concentration of the radical-monomer (IAm, for instance), whereas heating to 100° C. produces a significantly larger concentration of the radical monomer.

As various amounts of heat produces varying concentrations of free radicals, the concentration of antioxidant free radicals may be modulated by varying the amount of heating of the precursor compounds. Moreover, a person of skill in the art will appreciate that antioxidant precursor compounds comprising different moieties/monomeric units will have different labile bond strengths and will require a different temperature shift.

Reaction scheme 6 also illustrates the temperature dependent chemical equilibrium between the dimeric compound and its corresponding radical monomer. The temperature dependent equilibrium is responsible for the fact that a radical-dimer of formula I, formula II, formula III, or formula IV may be regenerated upon cooling Advantageously, the temperature dependent equilibrium allows one to design antioxidants with tailor-made properties, such as for example having a desired minimum activity at a specific temperature. The temperature dependent equilibrium may also enhance the lifetime and shelf life properties of an antioxidant according to the instant invention Temperature dependent absorption studies have been carried out in order to determine the bond dissociation energy (BDE) for the preferred dimers of formula IA, IB, IIA and IIIA. In particular, the monomer-radical resulting from the dissociation of each precursor compound has characteristic absorptions in the ultraviolet and visible regions. These absorptions are used to monitor the presence of the monomer-radicals in chemical equilibrium, such as for example the one illustrated at reaction scheme 5 for compound IA.

At a predetermined lower temperature the absorptions observed are different in comparison with the absorptions observed at the higher temperature ranges. However, when a sample of a compound of the invention is heated and subsequently cooled, the absorbance spectrum is similar if not identical before heating and after heating. These observations, together with other data, indicate that the dimer dissociation/formation is a reversible process.

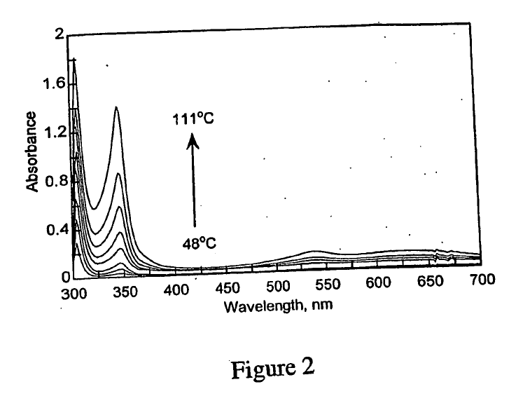

Referring now to FIG. 2, shown are the temperature dependent absorbance spectra of the monomer-radical obtained by thermal dissociation of compound IB in toluene. In particular, spectra were obtained for temperatures between 48° C. and 111° C. From the corresponding monomer absorbance spectra, values for the BDE of the C₁-C₁′ bond of compound IB were obtained. Data obtained from the temperature dependent study are analyzed using a simplified form of the Van't Hoff equation together with Beer's law. Accordingly, the experimental absorbance A is expressed as shown in equation 1:

$$ln A = \frac{ln ( {ɛ}^{2} {l}^{2} [ D ] )}{2} + \frac{Δ S}{2 R} - \frac{Δ H}{2 RT} \qquad ( 1 )$$

where ΔH, which corresponds to BDE, is obtained from the slope of an Arrhenius plot of lnA vs. 1/T. Similar temperature dependent studies were carried out to determine the BDE of compounds IA, IIA and IIIA. The experimentally obtained BDE values are tabulated in Table 1.

TABLE 1
Experimentally obtained BDE values.

| | monitored | temperature | C—C bond | EH_{diss} (kcal |
|---|---|---|---|---|
| radical | λₘₐₓ (nm) | range, ¼ C. | length (□) | mol⁻¹) |
| IB | 346 | 48 \check{S} 111 | 1.586 | 22.8 |
| IA | 346 | 37 \check{S} 100 | 1.596 | 23.6 |
| IIA | 336 | 48 \check{S} 102 | 1.608 | 26.2 |
| IIIA | 320 | 80 \check{S} 135 | — | 15.2 |

The data presented in Table 1 indicate that the BDE values of the compounds IA, IB, IIA, and IIIA are in a range between 15 and 26 kcal/mol, about four to five times lower than a value between 80 and 90 kcal/mol for a typical C—C bond. The data presented in Table 1 also indicates that the compounds according to the instant invention have different operating temperatures. For example, at 100° C. the compound according to formula IIIA is approximately 670,000 times more effective than the compound according to formula IIA in producing free radicals in the active form.

The apparent stability of the compounds in Table 1 is believed to be due to the intrinsic lack of reactivity toward oxygen, which makes the back reaction to reform the dimeric precursor compound the preferred reaction for the radical-monomer. Thus, the compounds of the present invention present the opportunity, at least in preferred embodiments, for repeated thermal cycling between a higher temperature and a lower temperature to thereby result in the formation of the antioxidant radicals (active) and antioxidant precursors (dormant) forms without significant degradation. Given the current knowledge of radical stabilities, a person of skill in the art will understand how to design materials in accordance with the instant invention that extend the BDE range above 26 kcal/mol, or below 15 kcal/mol.

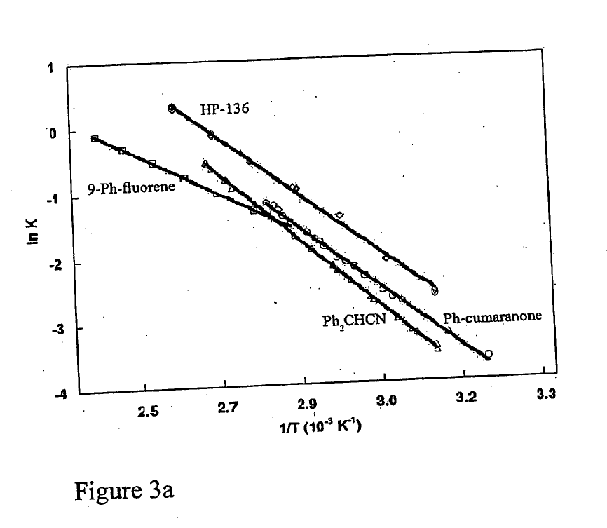

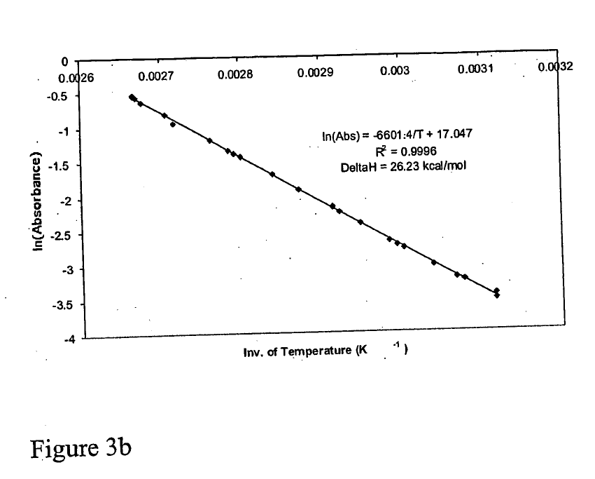

FIG. 3A shows Van't Hoff plots, according to equation 1, for thermally activatable antioxidant precursor compounds of formula IA, IB, IIA and IIIA, from which the corresponding BDE for compounds of formula IA, IIA and IIIA, were estimated and are listed in Table 1 above. While all plots gave excellent correlation, with statistical errors around ±0.1 kcal/mol, the inventors estimate that the true uncertainty of the BDE values is probably ±0.5 kcal/mol. FIG. 3B shows a further Van't Hoff plot, 336 nm, 48° C. to 102° C., 17.36 mM Diphehyl Acetonitrile under nitrogen.

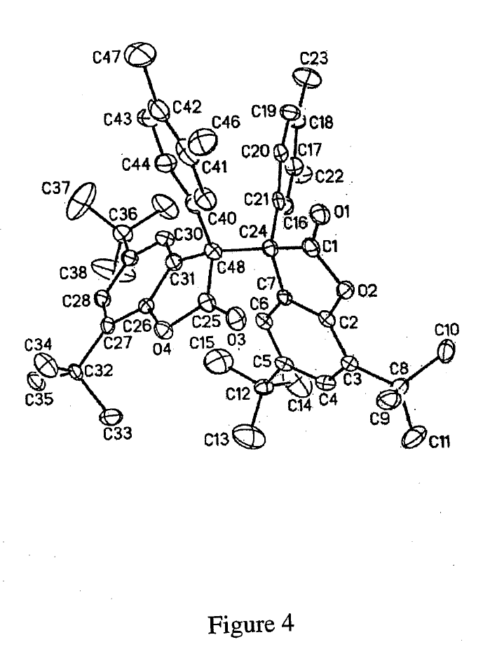

With reference to FIG. 4, a crystal structure is shown of a thermally activatable antioxidant precursor compound of the present invention. More specifically, a crystal structure of the compound of formula IB is shown (also disclosed in Frenette M., et al. “Bond dissociation Energies for Radical Dimers Derived from Highly Stablized Carbon-Centered Radicals” (2004) Organic Letters, 6(15), 2579-2582, which is incorporated herein by reference). Particular notice should be paid to Carbon-Carbon bond between C48 and C24, which represents the labile bond which is broken by dissociation of the moieties/monomeric units upon exposure to heat. The bond length of the C48-C24 bond is 1.586 Ångstroms (Å) which is approximately 0.05 Å longer than a average single bond between sp³ carbons, which is 1.54 Å. This significant lengthening is an indicator of the lowered BDE relative to typical Carbon-Carbon bonds and is a visual confirmation of the dissociation of the A-B precursor compound via breakage of the labile bond between moieties A and B upon heating of the antioxidant precursor compound.

Antioxidant precursor compounds and antioxidant free radicals of the present invention may be used to interrupt the oxidation chain of, for example, benzyl radicals. The antioxidant precursors and antioxidant free radicals of the present invention, however, may be used to reduce oxidation of any molecules, compounds or compositions, susceptible to oxidation. Preferably, the molecules, compounds or compositions are more susceptible to oxidation as the temperature increases. This allows one of skill in the art to use an antioxidant precursor of the present invention having a suitable BDE which ensures that a desired degree of dissociation of the precursor will take place below the oxidation temperature. In other words, when a thermal cycle is used, when heating the reaction mixture or target environment, the dormant precursor antioxidant compound will be activated before the oxidation temperature is reached thus preventing or slowing oxidation of the molecules, compound or composition susceptible to oxidation.

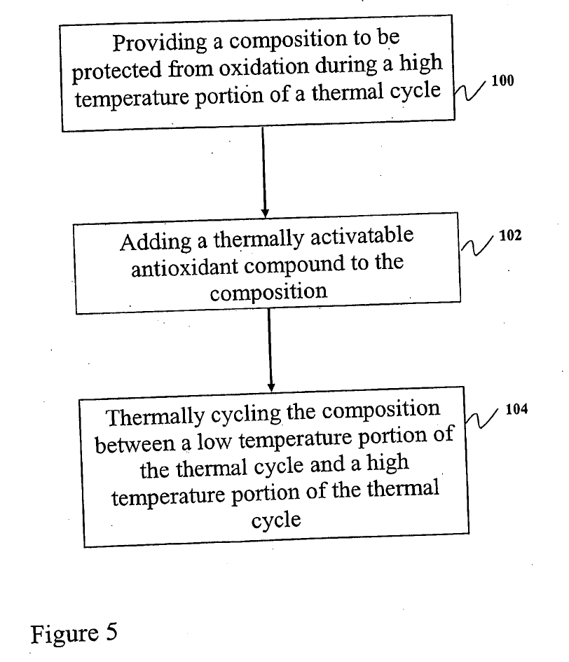

Referring now to FIG. 5, shown is a simplified flow diagram of one method of providing oxidation protection according to an embodiment of the instant invention, wherein the oxidation protection occurs during the high temperature portion of a high temperature/low temperature thermal cycle. At step 100, a composition to be protected against oxidation during the high temperature portion of the thermal cycle is provided. At step 102 an amount of a thermally activatable antioxidant precursor compound is added to the composition. At step 104, the composition is thermally cycled between the low temperature portion of the thermal cycle and the high temperature portion of the thermal cycle, causing at least partial dissociation and activation of the thermally activatable antioxidant precursor compound into the carbon-centered active antioxidant free radical species, so as to provide an increased amount of the carbon-centered active antioxidant free radical species during the high temperature portion of the thermal cycle relative to the low temperature portion of the thermal cycle. Preferably, the thermally activatable antioxidant precursor compound that is added at step 102 is one of the compounds of formula I, formula II, formula III, and formula IV.

Preferably, the antioxidant species that are utilized with the method according to FIG. 5 remain substantially in a dormant form until thermally activated, such as for instance by thermally cycling the composition between the low temperature portion of the thermal cycle and the high temperature portion of the thermal cycle. When the composition is maintained within the high temperature portion of the thermal cycle, the active antioxidant radicals scavenge or trap the radicals that mediate the oxidative degradation of materials. -In an exemplary embodiment of the present invention, when the composition, and therefore the active antioxidant radicals, are cooled, the active antioxidant species become dormant once again, and can be reused during subsequent thermal cycles.

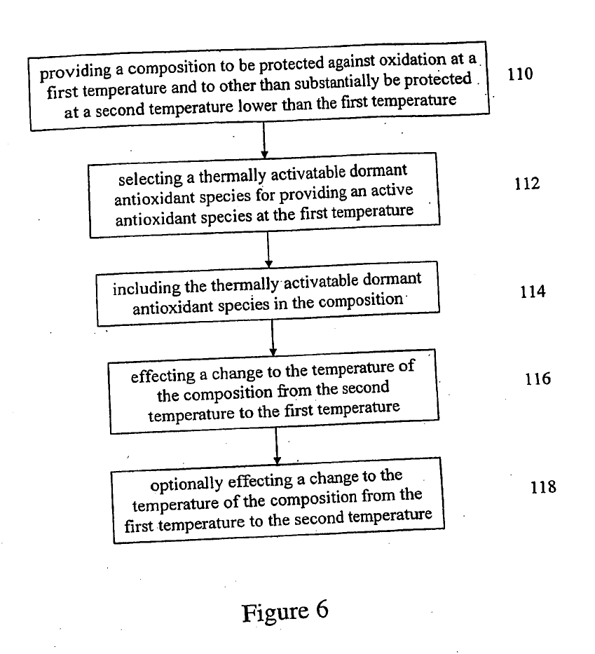

The method according to FIG. 6 is suited for use with compositions that may be subject to heat-cold cycles. For instance, some exemplary and non-limiting types of compositions include some plastics, lubricants, cooling fluids, and medical/pharmaceutical applications. Preferably, the method utilizes thermally activatable antioxidant precursor-compounds that have excellent fast response to repetitive hot-cold cycles, making the method suitable for use with systems that are subject to sudden, or “flash”, changes in temperature.

Referring now to FIG. 6, shown is a simplified flow diagram of a method of providing oxidation protection according to another embodiment of the instant invention. At step 110, a composition is provided that is to be protected against oxidation at a first temperature and that is to other than substantially be protected at a second temperature lower than the first temperature. At step 112, a thermally activatable antioxidant precursor compound is selected for providing an active antioxidant free radical at the first temperature, the thermally activatable antioxidant precursor compound thermally activatable at a temperature between the first and second temperatures. At step 114, the thermally activatable antioxidant precursor compound is included in the composition. At step 116, a change is effected to the temperature of the composition from the second temperature to the first temperature, to induce some of the thermally activatable antioxidant precursor compound into the active antioxidant species. At step 118, a change is effected to the temperature of the composition from the first temperature to the second temperature, to induce some of the active antioxidant species to regenerate the thermally activatable antioxidant precursor compound. Preferably, the thermally activatable antioxidant precursor compound that is included at step 114 is one of the compounds of formula IX or X as described herein.

Step 112 recites selecting a thermally activatable antioxidant precursor compound for providing an active antioxidant free radical at the first temperature. For instance, a particular thermally activatable antioxidant precursor compound having a known activation temperature or a known activation temperature range coinciding approximately with the first temperature is preferably selected from a plurality of available thermally activatable antioxidant precursor compounds. Preferably, the thermally activatable antioxidant precursor compound is selected from a list of available thermally activatable antioxidant precursor compounds, the list including data relating to activation temperature, solvent compatibility, recommended uses, etc. for a plurality of thermally activatable antioxidant precursor compounds. Optionally, the step of selecting includes a step of preparing a thermally activatable antioxidant precursor compound.

Preferably, the antioxidant precursor compounds utilized with the method according to FIG. 6 remain substantially in a dormant form, until thermally activated, such as for instance by thermally cycling the composition between the low temperature portion of the thermal cycle and the high temperature portion of the thermal cycle. When the composition is maintained within the high temperature portion of the thermal cycle, the active antioxidant free radicals generated scavenge the radicals that mediate the oxidative degradation of materials. When the composition, and therefore the active antioxidant free radicals, are cooled, the active antioxidant species become substantially dormant once again and thereby at least partially regenerate the thermally activatable antioxidant precursor compound, and can at least in preferred embodiments be reused during subsequent thermal cycles. The method according to FIG. 6 is well suited for use with compositions that may be subject to heat-cold cycles. For instance, some exemplary and non-limiting types of compositions include some plastics, lubricants, cooling fluids, and medical/pharmaceutical applications. Preferably, the method utilizes thermally activatable antioxidant precursor compounds that have excellent fast response to repetitive hot-cold cycles, making the method suitable for use with systems that are subject to sudden, or “flash”, changes in temperature.

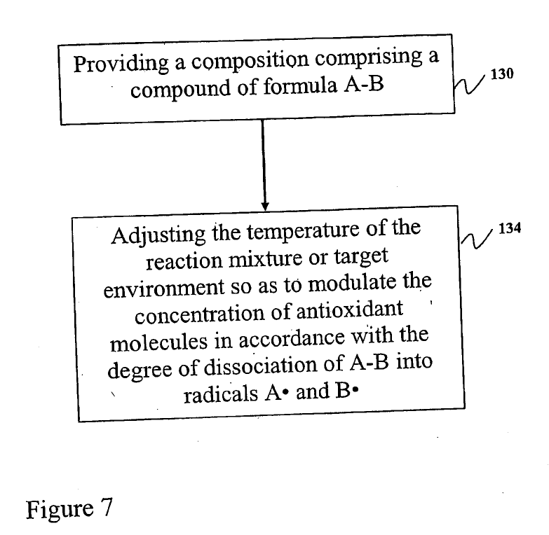

Another preferred method of the invention is illustrated with reference to FIG. 7 for generating an antioxidant. In FIG. 7 there is at step 130 provided a compound of formula A-B, according to the present invention as described above, wherein the compound A-B is substantially devoid of antioxidant properties, and is capable of dissociation into components A and B upon heating thereof, wherein at least one of A and B pertains to a radical exhibiting antioxidant activities. At step 134 the temperature of the reaction mixture or target environment is adjusted so as to modulate the concentration of antioxidant molecules in accordance with the degree of association or dissociation of A and B. The step of adjusting may pertain to a simple temperature shift, or in alternative embodiments may pertain to several temperature steps or continual temperature changes over a period of time, thereby to modulate and vary the concentration of active antioxidant species in the system.

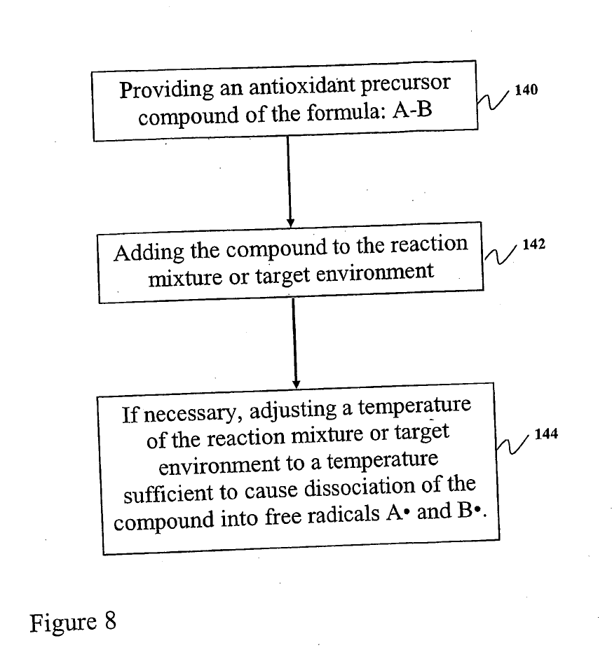

FIG. 8 illustrates another preferred method of the present invention for preventing or slowing oxidation of at least one molecule susceptible to oxidation in a reaction mixture or target environment. At step 140 the method includes providing an antioxidant precursor compound of the formula:

A-B

as described herein. Subsequent step 142 provides for adding the compound to the reaction mixture or target environment. Finally, step 144 provides, if necessary, for adjusting a temperature of the reaction mixture or target environment to a temperature sufficient to cause dissociation of the compound into free radicals A• and B•.

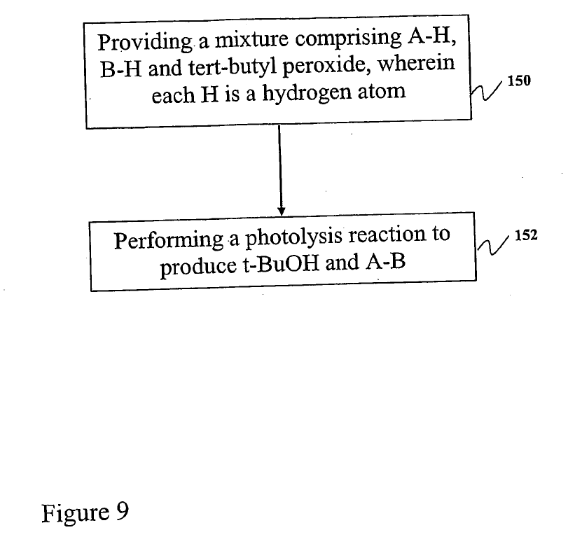

FIG. 9 illustrates yet another preferred method of the present invention for synthesizing a thermally activatable antioxidant precursor compound of the present invention. At step 150 there is provided a mixture comprising A-H, B—H and tert-butyl peroxide, wherein each H is a hydrogen atom. Subsequent step 152 involves performing a photolysis reaction to produce t-BuOH and A-B.

The combination of unidentical moieties/monomeric units to generate thermally activatable antioxidant precursor compounds of the present invention either before administration of the compounds or as a result of regeneration of the compounds following a thermal cycle is a further aspect of the present invention. Antioxidant precursor compounds comprising unidentical moieties/monomeric units may posses an advantage of generating a compound having a carbon-carbon labile bond with bond dissociation energy which differs to previous antioxidant precursor compounds. One of skill in the art will appreciate that this characteristic may be taken advantage of in a given situation where such variance is helpful or required.

Further, a composition comprising a number of various antioxidant precursor compounds of the present invention, either fully, partially or not dissociated into the corresponding antioxidant free radicals and one or more molecules susceptible to oxidation has been contemplated by the inventors and is another aspect of the present invention. A composition comprising one or more molecules having a variety of oxidization behaviour and/or characteristics, may benefit from exposure to a multitude of antioxidant precursor compounds and antioxidant free radicals of the present invention. The composition could then be treated with a thermal cycle or could simply be heated or cooled depending on the application.

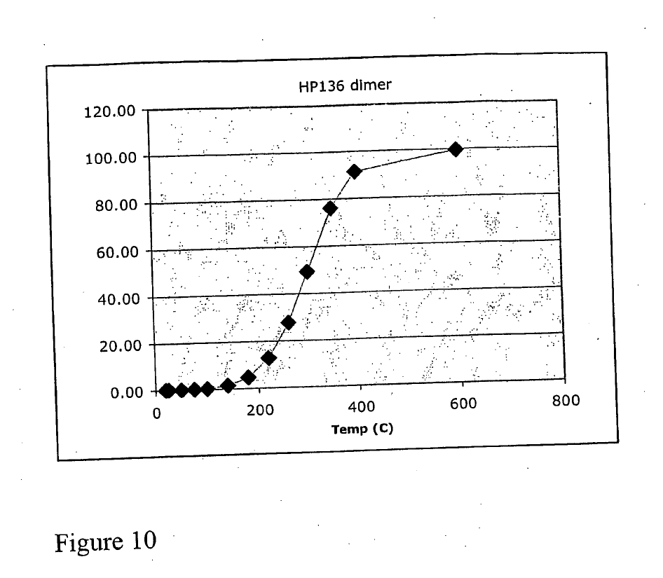

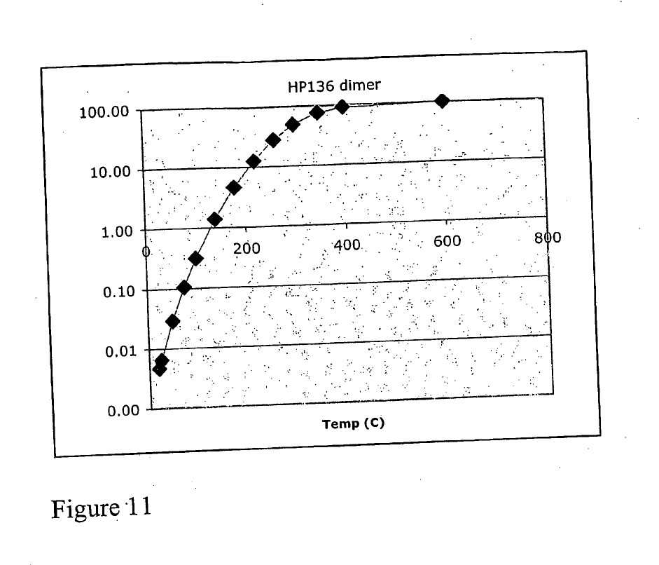

FIGS. 10 and 11 illustrate the degree of HP-136 (Ciba) dimmer dissociation at temperatures ranging from about 20° C. to about 400° C. Both graphs illustrate the degree of dimer dissociation based on 0.1% loading (y-axis) compared to temperature (x-axis), with FIG. 9 representing a log scale on the y-axis.

### PREPARATIVE EXAMPLES

¹H and ¹³C NMR spectra were recorded on a Bruker-Avance-300. spectrometer at 300 and 75.4 MHz, respectively. Mass spectrum (EI) was recorded on a Kratos-Concept-II instrument. Noncorrected melting point was determined on a Mel-Temp-II apparatus. The reaction was followed by TLC using silica gel 60 F₂₅₄ precoated 0.25 mm thick aluminum backed plates using short wave UV light for compound detection. Previous to use, tert-butyl peroxide was filtered through a plug of neutral aluminum oxide to remove eventual contents of water and tert-butanol.

### Preparative Example 1

### Preparation of the Compound According to Formula IA

The antioxidant precursor compound of formula IA was prepared by photolysis of di-tert-butyl peroxide in the presence of the corresponding monomer. A 500 mL Pyrex flask equipped with a magnetic stirring bar was used as the synthesis reactor. In the flask were placed 3-phenyl-isocoumaranone (10 g, 47.5 mmol), tert-butyl peroxide (100 mL, 544.3 mmol) and benzene (150 mL). The stirred solution was bubbled with nitrogen for 20 minutes and irradiated at 350 nm for 62 hours at 30° C. The irradiation dose in the UVA region of the spectrum (320 to 400 nm) was approximately 1.2 mJ/cm². The resulting suspension was concentrated using a rotatory evaporator. The solid residue was washed with cold diethyl ether until a white solid was obtained. The white solid was then recrystallized from diethyl ether to furnish 6.2 g of compound IA as a white solid (63% yield). ¹H NMR data match those previously reported. Mp 161-168 ° C. (decomposition from pink powder to dark red liquid), Rf=0.34 (hexanes/ethyl acetate=6:1), ¹H NMR (CDCl₃, 300 MHz): δ 7.38-7.18 (m, 12H), 7.09-7.01 (m, 4H), 6.46 (broad doublet, 2H, H4, H4′, J=6.4 Hz) ppm. ¹³C NMR (CDCl3, 75.4 MHz): δ 173.9, 153.8, 131.3, 131.2, 131.0, 130.7, 130.2, 129.3, 129.1, 128.7, 128.0, 127.7, 127.6, 126.8, 124.3, 124.0, 111.8, 111.2 ppm. MS (EI) m/z=209 [M/2]⁺(100%). An overview of this preparative method is shown in reaction scheme 7.

### Preparative Example 2

### Preparation of the Compound According to Formula IB

Synthesis of the antioxidant compound according to formula IB was carried out using the procedure reported for 3,3′-diphenyl-3H,3′H-[3,3′]bibenzofuranyl-2,2′-dione (IA). Mp 217-218° C. (decomposition from white powder to dark yellow liquid) ¹H NMR (CDCl₃, 500 MHz) δ 1.15 (s, 6 H), 1.30 (s, 6 H), 2.16 (s, 18 H), 2.26 (s, 18 H) 6.34 (broad s, 2 H), 7.02 (broad s, 4 H), 7.08 (broad s, 2 H), 7.25 (d, J=2 Hz, 2 H). ¹³C NMR δ 174.3 (s), 149.4 (s), 145.7 (s), 137.1 (s), 135.3 (s), 133.5 (s), 132.5 (d), 129.5 (s), 128.6 (d), 128.7 (d), 126.9 (s), 123.9 (d), 122.0 (s), 34.8 (s), 34.6 (s), 31.7 (q), 29.9 (q), 20.2 (q), 19.6 (q). MS m/z 349 (100), 334 (42), 307 (38), 291 (23).

### Preparative Example 3

### Preparation of the Compound According to Formula IIA

The antioxidant precursor compound according to formula IIA was prepared by adding to 100 mL of tert-butyl peroxide and 10 mL HMPA, diphenylacetonitrile (10 g, 50 mmol). The resulting solution (in a Pyrex bottle) was bubbled with nitrogen for 30 minutes. Irradiation was done with 350 nm lamps and followed by TLC for 8 days. (Note: reaction times vary with intensity of light). The reaction mixture was then reduced in volume and the residue was recrystallized in diethyl ether to afford 2.8 g of white crystals. (28% yield) mp 192-203° C. (decomposition from yellow powder to dark orange liquid, ¹H NMR (CDCl3, 300 MHz): δ 7.20-7.35 (broad m) ppm. ¹³C NMR (CDCl3, 75.4 MHz): δ 137.3, 130.5,129.0,128.5, 121.5,59.5 ppm. MS (EI) m/z 192 [M/2]⁺ (100%). Note: This reaction has recently been scaled up to work with 50 g starting material with yields as high as 65% using optimized conditions. An overview of this preparative method is shown in reaction scheme 8.

### Preparative Example 4

### Preparation of the Compound According to Formula IIIA

The antioxidant precursor compound according to formula IIIA was obtained in a one-pot synthesis. 9-Phenyl-9-fluorenol (1.0 g, 3.87 mmol) was dissolved in dry, freshly distilled acetone (over CaH₂). This solution was added drop-wise to a solution of previously reacted trimethylsilyl chloride (0.51 mL, 1.2 eq.) and excess NaI (2.9 g, 5 eq.) in acetone, all under inert atmosphere (argon) at room temperature. The solution turned brown from the initial addition and darkened as the reaction was stirred overnight (12 hrs). After evaporation by rotovap, the residue was washed between dichloromethane (100 mL) and 100 mL of 10% Na₂S₂O₃(aq.) (the aqueous phase was clear and the dichloromethane layer was opaque with a white suspension). Simple filtration of this bilayer afforded the desired product (which was washed several times with ice cold acetone) to afford 480 mg of product as a white powder. (51% yield) mp 176-185° C. (decomposition from pink powder to dark red liquid), ¹H NMR (CDCl₃, 200 MHz): δ 7.59-7.46 (6H), 7.30-7.09 (m,16H), 6.93 (broad s, ˜2H), 6.57 (broad s, ˜2H) ppm. ¹³C NMR: δ 147.5, 146.3, 141.5, 140.9, 131.5, 129.4, 129.2, 128.3, 127.7, 127.6, 127.6, 127.3, 127.2, 126.7, 126.4, 126.0, 119.8, 119.5, 93.5 ppm. MS (EI) m/z 241 [M/2]⁺ (100%). HRMS calculated for C₁₉H₁₃⁺ (M/2⁺) 241.1012, found 241.1037. An overview of this preparative method is shown in reaction scheme 9.

### Temperature Dependent UV-VIS Measurements

A typical experiment was conducted as follows: A quartz cell containing ca. 20 mg of the dimer in 3.0 mL of dry toluene (or in 1,3-dichlorobenzene in the case of antioxidant precursor compound of formula IIIA) was capped with a septum, degassed under nitrogen flow and was connected to a nitrogen filled balloon (to accommodate gas expansion during heating). A thermocouple probe was passed through the septum directly in the solution to measure the exact temperature S3 of the solution at all times during the experiment. The heating was achieved with a custom designed, controllable block heater that fit in the spectrophotometer cavity. Room temperature absorbance spectra were set as the baseline. Absorbance measurements were taken every 3-5 degrees with at least 5 minutes in between measurements to allow the temperature and the solution to equilibrate.

Shown as reaction schemes 7, 8 and 9 is a brief overview of an exemplary synthesis using the methods described in preparative examples 1, 3 and 4, of some of the thermally activatable antioxidant precursor compounds according to the present invention.

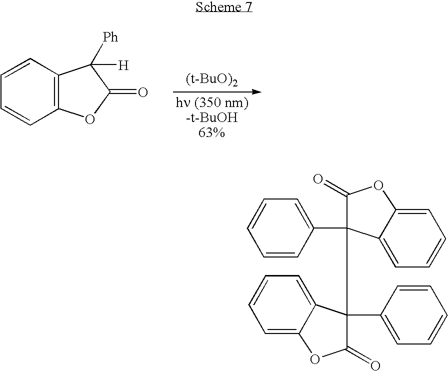

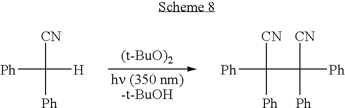

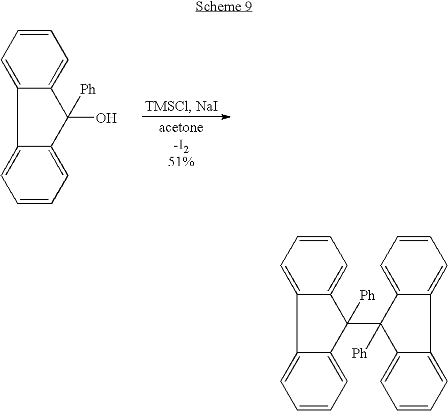

The thermally activated dormant antioxidants according to the instant invention are preferably usable to protect materials that are subjected to heat-cold cycles, including plastics, lubricants, cooling fluids, and medical or pharmaceutical applications. In preferred embodiments of the present invention, the compounds disclosed are suitable for use in situations in which sudden flash changes in temperature occur. Potential fields of applications include oils in closed systems that are subjected to heating, oils in agricultural machines that circulate into a high temperature environment for a brief period, thermo-set resins, thermoplastic resins, and protective films subjected to heat, among others.

Optionally the thermally activated dormant antioxidants may be used in conjunction with a “common” antioxidant, such as for example an anti-oxidant that provides protection at room temperature, so as to provide anti-oxidant protection over a wide range of temperatures.

Numerous other embodiments may be envisaged without departing from the spirit and scope of the invention.

1. A thermally activatable antioxidant precursor compound of the formula:
A-B
wherein A and B are the same or different, each consisting of a moiety other than a hydrogen atom; and
wherein A and B are connected via a labile bond, and are able to dissociate through breakage of the labile bond upon exposure of said compound to a predetermined temperature shift from a lower temperature to a higher temperature, thereby to generate corresponding free radicals A• and B• at least one of which being suitable for use as an antioxidant.

2. The thermally activatable antioxidant precursor compound of claim 1, wherein each of A and B comprises a monocyclic aromatic or polycyclic aromatic ring system, optionally substituted at one or more positions and optionally comprising a heterocyclic ring.

3. The thermally activatable antioxidant precursor compound of claim 1, wherein the labile bond is a carbon-carbon bond and each of the free radicals As and Be is a carbon centered free radical.

4. (canceled)

5. (canceled)

6. (canceled)

7. The thermally activatable antioxidant precursor compound of claim 1, wherein the free radicals A• and B• are able to re-associate through the formation of a labile bond upon exposure of said radicals to a predetermined temperature shift from a higher temperature to a lower temperature thereby to regenerate the corresponding antioxidant precursor compound of the formula:
A-B

8. (canceled)

9. (canceled)

10. The thermally activatable antioxidant precursor compound of claim 1, wherein the compound A-B is selected from a compound of the formula X: 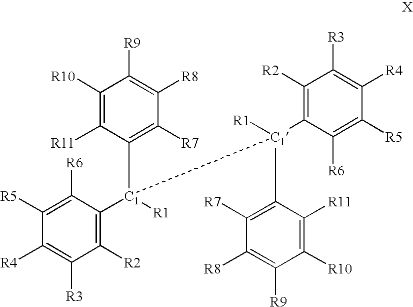
wherein the dashed line represents the labile bond susceptible to breakage upon exposure of said compound to a temperature shift from a lower temperature to a higher temperature; and wherein R1-R11 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl,
and wherein optionally any two of R2 to R11 within the same moiety of A or B may be linked to form a substituted or unsubstituted bicyclic or polycyclic fused ring system, and wherein optionally R1 may be linked to one or more of R2, R6, R7, and R11 within the same moiety of A or B, to form a substituted or unsubstituted bicyclic or polycyclic fused ring system optionally comprising one or more heterocyclic rings;
or a compound of formula I: 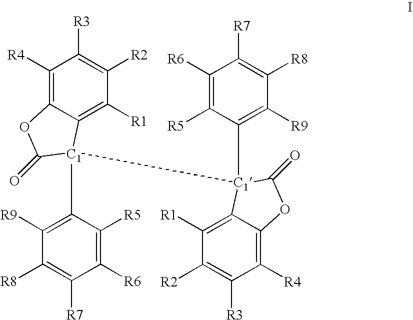
wherein the dashed line represents the labile bond susceptible to breakage upon exposure of said compound to a temperature shift from a lower temperature to a higher temperature: and wherein RI to R9 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F) CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-₁₈ alkyl, and C₆-C₂₀ aryl, optionally substituted with one or more C₁-C₁₈ alkyl;
or a compound of formula II: 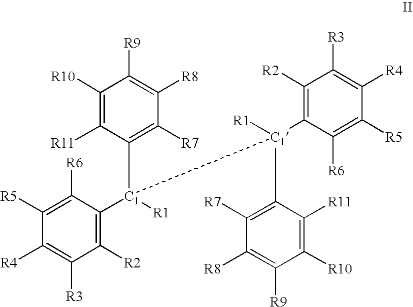
wherein the dashed line represents the labile bond susceptible to breakage upon exposure to a higher temperature, and
wherein R1 represents an electron withdrawing group optionally selected from CN, CHal₃ (where Hal=Cl, Br or F), CO₂R16 (where R16 comprises hydrogen or a substituent selected from a linear or branched C₁-C₁₈ alkyl linear or branched C₂-C₁₈ alkenyl, linear or branched C₂-C₁₈ alkynyl, C₅-C₈ cycloalkyl, and C₆-C₂₀ aryl), and NO₂;
R2-R11 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂aryl), NO₂, C₅-C₈ cycloalkyl optionally substituted with one or more C₁-C₁₈ alkyl, and C₆-C₂₀ aryl, optionally substituted with one or more C₁-C₁₈ alkyl; or a compound of formula III: 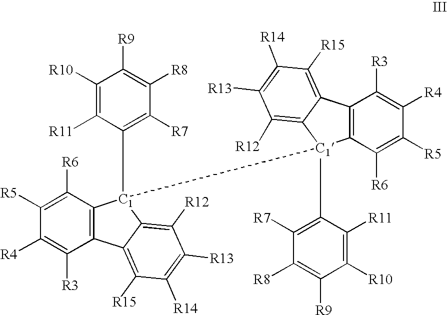
wherein the dashed line represents the labile bond susceptible to breakage upon exposure to a higher temperature; and
wherein R3-R15 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₈ alkyl; or a compound of formula IV: 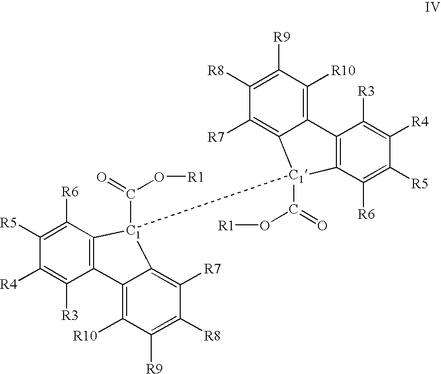
wherein the dashed line represents the labile bond susceptible to breakage upon exposure to a higher temperature, and
wherein R1 and R3-R10 are the same or different, each independently selected from hydrogen or a substituent selected from the following group: linear or branched C₁-C₁₈ alkyl, linear or branched C₂-C₁₈ alkenyl, linear or branched C₂-C₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl, or
a compound of formula IA: 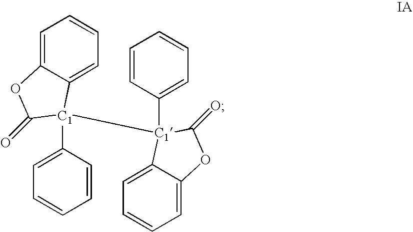
or a compound of formula IB: 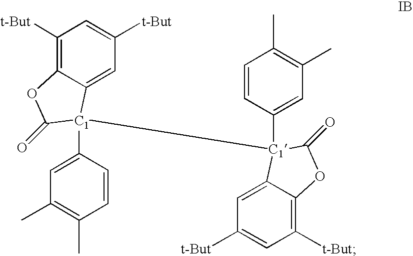
or a compound of formula IIA: 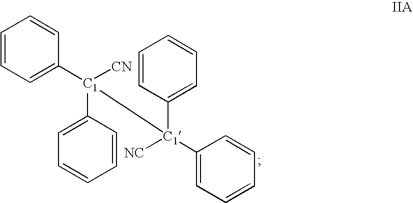
or a compound of formula IIIA: 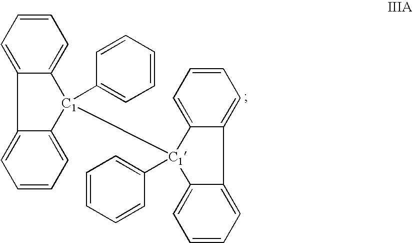
or a compound of formula IVA:

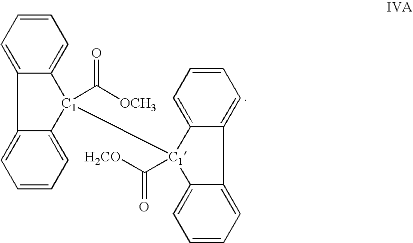

11. (canceled)

12. (canceled)

13. (canceled)

14. (canceled)

15. (canceled)

16. (canceled)

17. (canceled)

18. (canceled)

19. (canceled)

20. (canceled)

21. The thermally activatable antioxidant precursor compound of claim 1, wherein one of or both of the free radicals A• and B• are of the formula V: 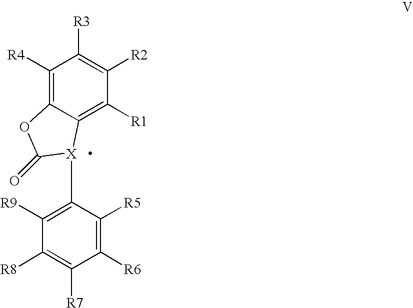
wherein X represents C₁ or C₁′ and R1 to R9 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl;
or one of or both of the free radicals A• and B• are of the formula VI: 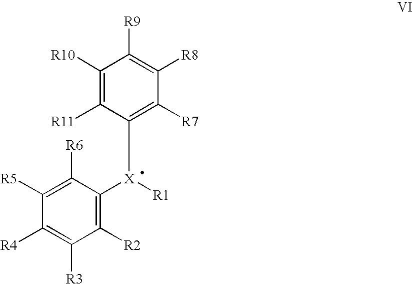
wherein X represents C₁ or C₁′ and R1 represents an electron withdrawing group selected from CN, CHal₃ (where Hal=Cl, Br or F), CO₂R16 (where R16 comprises hydrogen or a substituent selected from a linear or branched C₁-C₁₈ alkyl, linear or branched C₁-C₁₈ alkenyl, linear or branched C₂-C₁₈ alkynyl, C₅-C₈ cycloalkyl optionally carrying C₁-C₁₈ alkyl groups, and C₆-C₂₀ aryl), and NO₂;
and R2 to R11 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl) NO₂, C₅-C₈ cycloalkyl optionally substituted with one or more C₁-C₁₈ alkyl, and C₆C₂₀ aryl, optionally substituted with one or more C₁-C₁₈ alkyl;
or one of or both of the free radicals A• and B• are of the formula VII: 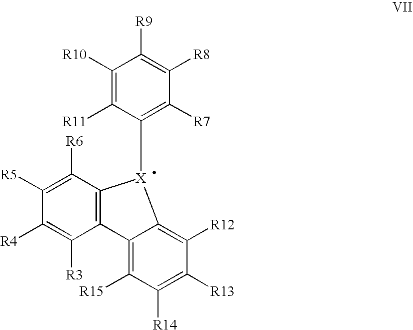
wherein X represents C₁ or C₁′ and R3 to R15 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl;
or one of or both of the free radicals A• and B• are of the formula VIII: 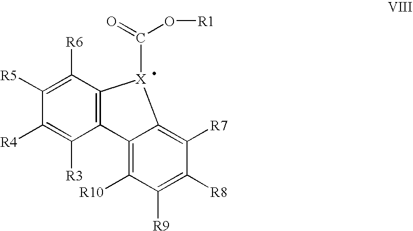
wherein X represents C₁ or C₁′ and R1 and R3 to RIO are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl and C₆-C₂₀ aryl), NO₂, C₅-C₈ cycloalkyl optionally substituted with one or more C₁-C₁₈ alkyl, and C₆C₂₀ aryl, optionally substituted with one or more C₁-C₁₈ alkyl;
or one or both of the free radicals A• and B• are of the formula VA: 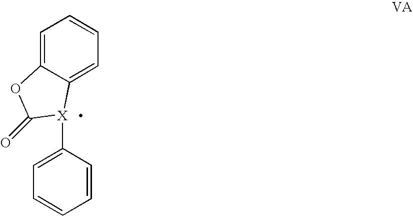
wherein X represents C₁ or C₁′;
or either or both of the free radicals As and Be are of the formula VB: 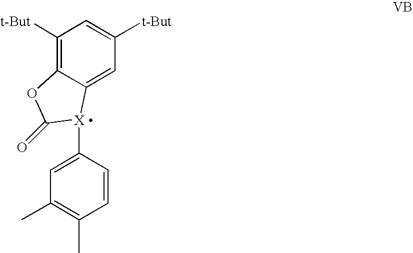
wherein X represents C₁ or C₁′;
or either or both of the free radicals A• and B• are of the formula VIA: 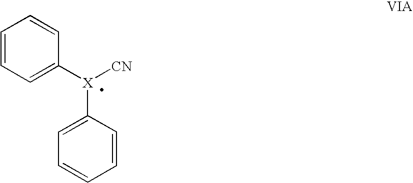
wherein X represents C₁ or C₁′;
or either or both of the free radicals A• and B• are of the formula VIIA: 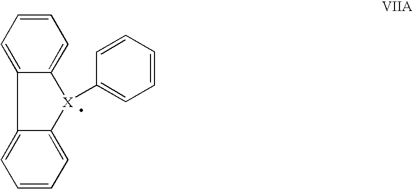
wherein X represents C₁ or C₁′;
or either or both of the free radicals A• and B• are of the formula VIIIA: 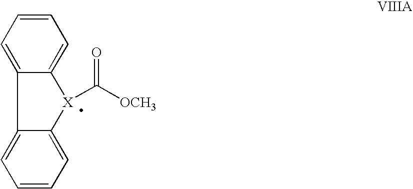
wherein X represents C₁ or C₁′,
or at least one of Ax and Be is selected from the group consisting of: 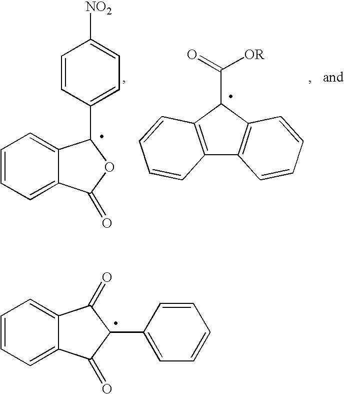
wherein R is selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₁-C₈ cycloalkyl, and C₆-C₂₀ aryl), NO₂, C₅-C₈ cycloalkyl optionally substituted with one or more C₁-C₁₈ alkyl, and C₆-C₂₀ aryl, optionally substituted with one or more C₁-C₁₈ alkyl;
or one of or both of the free radicals A• and B• are of the formula IX: 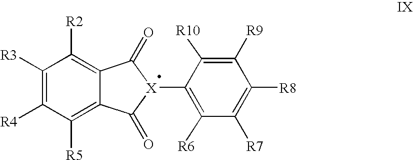
wherein X represents C₁ or C₁′, and R1 to R10 are the same or different, each independently selected from hydrogen or a substituent selected from the following group: linear or branched C₁-C₁₈ alkyl, linear or branched C₂-C₁₈ alkenyl, linear or branched C₂-C₁₈ alkynyl, CN, CHal₃ (where Hal=Cl, Br or F), CO₂R16 (where R16 comprises hydrogen or a substituent selected from a linear or branched C₁-C₁₈ alkyl, linear or branched C₂-C₁₈ alkenyl, linear or branched C₂-C₁₈ alkynyl, C₅-C₈ cycloalkyl, and C₆-C₂₀ aryl), NO₂, C₅-C₈ cycloalkyl optionally substituted with one or more C₁-C₁₈ alkyl and C₆-C₂₀ aryl, optionally substituted with one or more C₁-C₁₈ alkyl.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. The thermally activatable antioxidant precursor compound of claim 1, with the proviso that the compound is not the compound of formula (11) disclosed in U.S. Pat. No. 5,367,008 or the compound of formula (10) disclosed in U.S. Pat. No. 5,428,177 or the dimeric form of Irganox HP-136 (Ciba Specialty Products) or related dimeric products.

34. The thermally activatable antioxidant precursor compound of claim 1, wherein the bond dissociation energy of the labile bond is less than 80 kcal/mol preferably less than 50 kcal/mol, more preferably less than 25 kcal mol, most preferably less than 20 kcal/mol.

35. (canceled)

36. (canceled)

37. (canceled)

38. The thermally activatable antioxidant compound of claim 1, wherein said temperature shift is from a lower temperature of from 0° C. to 40° C. to a higher temperature of from 20° C. to 400° C.

39. Use of a compound of the formula:
A-B
as defined in of claim 1, as a thermally activatable antioxidant precursor compound.

40. A composition comprising: (a) a compound susceptible to oxidation; and (b) the thermally activatable antioxidant precursor compound of claim 1, wherein the compound (a) is optionally more susceptible to oxidation at a higher temperature than at a lower temperature.

41. (canceled)

42. A method for generating an antioxidant, the method comprising the steps of:
a) providing an antioxidant precursor compound of the formula:
A-B
as described in of claim 1; and
b) adjusting the temperature of A-B to thereby cause dissociation of the compound into free radicals A• and B• at least one of which is suitable for use as an antioxidant.

43. The method of claim 42, wherein the step of adjusting comprises shifting the temperature of the antioxidant precursor compound from a lower temperature of from 0° C. to 40° C. to a higher temperature of from 20° C. to 400° C.

44. (canceled)

45. The method of claim 43 wherein the antioxidant precursor compound can be at least in part reformed at said lower temperature; step b) comprising heating the antioxidant precursor compound to said higher temperature, the method further comprising step c) cooling the free radicals A• and B• formed in step b) to said lower temperature to thereby cause at least partial reassociation of the free radicals Ad and Be into the thermally activatable antioxidant precursor compound A-B.

46. (canceled)

47. A method of preventing or slowing oxidation of at least one molecule susceptible to oxidation in a reaction mixture or target environment, the method comprising the steps of:
a) providing an antioxidant precursor compound of the formula:
A-B
as described in claim 1;
b) adding the compound to the reaction mixture or target environment; and
c) if necessary adjusting a temperature of the reaction mixture or target environment to a temperature sufficient to cause dissociation of the compound into free radicals A• and B•.

48. The method of claim 47, wherein the step of adjusting comprises shifting the temperature of the antioxidant precursor compound from a lower temperature of from 0° C. to 40° C. to a higher temperature of from 20° C. to 400° C.

49. (canceled)

50. (canceled)

51. The method of claim 48, wherein the method involves a thermal cycle comprising a lower temperature portion following a higher temperature portion, the method further comprising step d) cooling the reaction mixture or target environment to the lower temperature portion of the thermal cycle thereby to cause at least partial reassociation of A and B to form the thermally activated antioxidant precursor compound A-B.

52. (canceled)

53. A composition comprising at least one molecule susceptible to oxidation, and two or more compounds according to claim 1, each of said two or more compounds having alternative combinations of moieties A and B.

54. The composition of claim 53, wherein each of said two or more compounds comprises a labile bond having a bond strength that is different from all other compounds of said two or more compounds.

55. (canceled)

56. A method for synthesizing the thermally activatable antioxidant precursor compound of claim 1, the method comprising the steps of:
a) providing a mixture comprising A-H, B—H and tert-butyl peroxide, wherein each H is a hydrogen atom and each moiety A and B is the same or different; and
b) performing a photolysis reaction, preferably at about 350 nm to produce t-BuOH and A-B.

57. (canceled)

58. (canceled)
